SEQUENCE LISTING (16 sequences)

SEQ ID NO: 1 - protein, 5 aa, synthetic construct
GGGGG

SEQ ID NO: 2 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 3 - protein, 58 aa, synthetic construct
AEAKYAKEKIFAVGEIYWLPNLTHGQIMAFIAALNDDPSQSSELLSEAKKLNDSQAPK

SEQ ID NO: 4 - protein, 58 aa, synthetic construct
AEAKYAKEKIIALSEIIWLPNLTHGQIMAFIAALNDDPSQSSELLSEAKKLNDSQAPK

SEQ ID NO: 5 - protein, 58 aa, synthetic construct
AEAKYAKEKIAALSEIIWLPNLTHGQIMAFIAALNDDPSQSSELLSEAKKLNDSQAPK

SEQ ID NO: 6 - protein, 58 aa, synthetic construct
AEAKYAKEKVHALSEIIWLPNLTHGQIMAFIAALNDDPSQSSELLSEAKKLNDSQAPK

SEQ ID NO: 7 - protein, 5 aa, synthetic construct
GGGGS

SEQ ID NO: 8 - protein, 9 aa, synthetic construct
GGGGGGGGG

SEQ ID NO: 9 - protein, 9 aa, synthetic construct
EEEEEEEEE

SEQ ID NO: 10 - protein, 9 aa, synthetic construct
SSSSSSSSS

SEQ ID NO: 11 - protein, 9 aa, synthetic construct
GGGGGCPPC

SEQ ID NO: 12 - protein, 15 aa, synthetic construct
GGGGSGGGGSGGGGS

SEQ ID NO: 13 - protein, 13 aa, synthetic construct
SCVPLMRCGGCCN

SEQ ID NO: 14 - protein, 14 aa, synthetic construct
PSCVPLMRCGGCCN

SEQ ID NO: 15 - protein, 9 aa, synthetic construct
GDLIYRNQK

SEQ ID NO: 16 - protein, 23 aa, synthetic construct
GGGGGGGGGPSCVPLMRCGGCCN

(19) United States
(12) Patent Application Publication
(10) Pub. No.: US 2024/0024503 A1
(43) Pub. Date: Jan. 25, 2024

(54) METHODS AND COMPOSITIONS FOR PRODUCING TARGETED MICROBUBBLES

(71) Applicant: The Board of Trustees of the Leland Stanford Junior University, Stanford, CA (US)

(72) Inventors: Rakesh Bam, Menlo Park, CA (US); Jeremy Dahl, Palo Alto, CA (US)

(21) Appl. No.: 18/352,081

(22) Filed: Jul. 13, 2023

Related U.S. Application Data
(60) Provisional application No. 63/389,240, filed on Jul. 14, 2022.

(51) Int. Cl. A61K 47/69; C07K 16/28; A61K 45/06

(52) U.S. Cl. CPC A61K 47/6909; C07K 16/2827; A61K 45/06; B82Y 5/00

(57) ABSTRACT

Methods and compositions for the production of phospholipid-ligand bioconjugates and uniform targeted microbubbles are provided. These methods and compositions find use in ultrasound and molecular imaging applications related to cancer and other diseases. The methods of the present disclosure comprise contacting a phospholipid comprising a maleimide containing functional group with a ligand comprising a C terminal cysteine residue. The methods disclosed herein solves the problems in producing ready-to-use and clinically translatable ultrasound molecular imaging agents by incorporating small protein ligands engineered to bind against biomarkers representing pathological angiogenesis or abnormal cells. The methods also overcome the current limitations in producing uniformly targeted microbubbles in a scalable, economical and reproducible manner.

### CROSS-REFERENCE TO RELATED APPLICATIONS

Pursuant to 35 U.S.C. § 119 (e), this application claims priority to the filing date of U.S. Provisional Patent Application Ser. No. 63/389,240 filed Jul. 14, 2022, the disclosure of which application is herein incorporated by reference.

### GOVERNMENT RIGHTS

This invention was made with Government support under contract CA218204 awarded by the National Institutes of Health. The Government has certain rights in the invention.

### SEQUENCE LISTING

A Sequence Listing is provided herewith as a txt file Sequence Listing XML, “STAN-2001_Seq_List” created Jul. 13, 2023 and having a size of 15,697 bytes. The contents of the txt file Sequence Listing XML are incorporated by reference herein in their entirety.

### BACKGROUND OF THE INVENTION

Molecularly targeted contrast agents are desirable for high-specificity and high-sensitivity medical imaging techniques. Phospholipids-based microbubbles (MBs) serve as blood pool contrast agents for contrast-enhanced ultrasound imaging. These MBs have a chemically inert gas (sulfur hexafluoride, perfluorocarbons, etc.) core encapsulated within a phospholipid monolayer. Target-specific antibodies or engineered small protein ligands can be assembled at the liquid interface of the phospholipid-monolayer shell by chemical conjugation methods to generate contrast agents for the ultrasound molecular imaging of disease processes. Although many techniques are available for targeted MBs preparations, a standardized and reproducible methodology for protein ligand design, phospholipid-ligand bioconjugation, and targeted MB production via microfluidics will accelerate the clinical translation of new imaging agents by providing an optimal platform technology.

Engineering of clinically translatable lipid-coated microbubbles adopts the paradigm of composition, processing, structure, property and performance. Currently, there are no FDA-approved targeted MBs for ultrasound molecular imaging; moreover, the FDA-approved non-targeted MBs are mainly produced by mechanical agitation methods, which produces MBs of broader size distribution, and can cause heterogeneous acoustic response and variabilities in imaging sensitivity under ultrasound. Microfluidic-based processing methods offer better control over MB size dispersity for molecular imaging applications as well as production efficiency and reproducibility. Microfluidic devices produce MBs by a variety of pinch-off mechanisms with precisely controlled flow of gas and liquid streams. Chips fabricated with specific microfluidic channel geometry can consistently maintain production uniformity of MBs compared to those generated using sonication/amalgamation techniques. Peyman et al. reported that MBs produced by a flow-focusing microfluidic device have higher ultrasound scattering properties over a wide range of frequencies compared to those formed by mechanical agitation methods. Consistently, microfluidic techniques can be applied to produce uniform suspensions of MBs with contrast properties tailored to specific bandwidths of ultrasound imaging. For the contrast-enhanced molecular imaging applications, labeling consistency of ligands on the MB shell is important for the optimal target-binding performance of targeted MBs. Furthermore, ligand labeling distribution can be enhanced upon preferential use of phospholipid formulations and specific lipid handling methods prior to MB production. Microfluidic devices may allow precise control over these production parameters to achieve standardized methodologies of targeted MB synthesis.

Provided herein are methods and compositions for the production of uniformly targeted microbubbles in a scalable, economical and reproducible manner.

### SUMMARY OF THE INVENTION

Methods and compositions for the production of phospholipid-ligand bioconjugates and uniform targeted microbubbles are provided. These methods and compositions find use in ultrasound and molecular imaging applications related to cancer and other diseases. The present disclosure includes a method of producing a phospholipid-ligand bioconjugate, the method comprising contacting a phospholipid polymer comprising a maleimide-containing functional group, with a ligand comprising a C terminal cysteine residue. The methods disclosed herein solves problems in producing ready-to-use and clinically translatable ultrasound molecular imaging agents, by incorporating small protein ligands engineered to bind biomarkers associated with pathological angiogenesis or abnormal cells. The methods also overcome the current limitations in producing uniformly targeted microbubbles in a scalable, economical and reproducible manner.

For example and without limitation, it is shown herein that affibodies modified with C terminal cysteine residues were screened for binding affinity for B7-H3, an immune checkpoint protein expressed on cancer and vascular endothelial cells. The cysteine-maleimide conjugation approach allows for site-specific conjugation and site-specific bioconjugation with a terminal cysteine and reduces the possibilities for altered protein activity that may, otherwise, arise due to random conjugations at multiple available amines in proteins. The affibody with the highest affinity for B7-H3 was selected and conjugated to the phospholipid polymer 1,2-distearoyl-sn-glycero-3-phosphoethanolamine-N-[maleimide(polyethylene glycol)-2000] (ammonium salt) (DSPE-PEG[2000]-Mal) utilizing thiol-Michael addition reactions thereby forming phospholipid-ligand conjugate micelles. The phospholipid-ligand conjugate micelles were mixed with 1,2-dipalmitoyl-sn-glycero-3-phosphocholine (DPPC) liposomes in the presence of perfluorobutane gas using a microfluidic device. The resulting targeted microbubbles were tested for binding to endothelial cells expressing B7-H3 in vitro and mouse mammary tumors expressing B7-H3 in vivo. Targeted microbubbles specifically bound to cells expressing B7-H3, whereas control microbubbles lacking the affibody bound at significantly fewer numbers relative to the targeted microbubbles.

In some embodiments, the method of producing the phospholipid-ligand bioconjugate comprises heating the phospholipid polymer comprising a maleimide containing functional group to greater than 50° C., for at least about 2 hours, then reducing the temperature of the phospholipid polymer to room temperature for at least 1 hour prior to the contacting step. The phospholipid solution is maintained above its critical micellar concentration.

Phospholipid-ligand bioconjugates of the present disclosure may be comprised of a range of different phospholipid polymers and ligands (e.g. affibodies). Phospholipid polymers that find use in the present disclosure include, without limitation, DSPE-PEG(2000), DSPE-PEG(2000)-Mal, 1,2-distearyol-sn-glycero-3-phosphoethanolamine-N-[amino(polyethylene glycol) 2000](DSPE-PEG2000-A), 1,2-distearyol-sn-glycero-3-phosphoethanolamine-N-[biotinyl (polyethylene glycol)2000] (DSPE-PEG2000-B) and 1,2-distearyol-sn-glycero-3-phosphoethanolamine-N-[methoxy(polyethylene glycol) 5000] (DSPE-PEG5000), etc.

Ligands for use in the present disclosure include any ligand that binds to a protein, lipid or small molecule found on the surface of a cell. Ligands that find use in the present disclosure include, without limitation, affibodies, single-chain variable fragments, single-domain antibodies, diabodies, etc. The ligands may bind to any protein deemed useful. Proteins of particular interest include proteins that are specifically expressed on cancer and endothelial cells (e.g. B7-H3). In some embodiments, the ligand is an AC12 affibody. All ligands of the present disclosure comprise a C terminal cysteine residue or are engineered to comprise a C terminal cysteine. In some embodiments, the C terminal cysteine is preceded by a pentaglycine bridge.

The phospholipid polymers of the present disclosure are contacted with the ligand under specific conditions. In some embodiments, the phospholipid is contacted with the ligand in a ratio of from about 10:1 to about 30:1, e.g. at about 20 to 1 ratio (20 phospholipid to 1 ligand). In some embodiments, the contacting step is performed at a neutral pH, e.g. from about pH 6.8 to about pH 7.2.

In another embodiment, a method is provided for producing targeted microbubbles. In these embodiments, the phospholipid-ligand conjugates of the present disclosure are contacted with a phospholipid liposome in the presence of an inert gas. In some embodiments, two or more different micelles comprising phospholipid-ligand bioconjugates are generated for use in a diagnostic composition. For instance, there may be a first phospholipid-ligand bioconjugates that has a ligand that targets a first protein and a second phospholipid-ligand bioconjugates that has a ligand that targets a second protein that is different from the first protein.

A range of different phospholipid liposomes can be used in the production of the targeted microbubble including, without limitation, DPPC, DSPC, DPPA, DPPG etc. The inert gas forms a gas core within the targeted microbubble. The gas core determines the echogenecity of the microbubble in ultrasound applications. Any inert gas may be used to provide the desired echogenicity. Inert gases of the invention include, without limitation, perfluorobutane, octafluoropropane, perfluorocarbon, sulfur hexafluoride, helium, neon, argon, krypton, xenon, radon, oganesson, etc.

The targeted microbubbles of the present disclosure may comprise specific amounts of phospholipid-ligand conjugates and phospholipids. In some embodiments, the targeted microbubbles comprise 1-10 mole percent phospholipid-ligand conjugate. In some embodiments, the targeted microbubble comprises 90-99 mole percent phospholipid.

Prior to the addition of the phospholipid liposome to the phospholipid-ligand conjugate micelles, the phospholipid liposome may have a specific diameter. Phospholipid liposomes may be prepared by sonication or extrusion methods. In some embodiments, the diameter of the phospholipid liposome is at least about 100 nm. The targeted microbubbles may also have specific size characteristics. In some embodiments, the targeted microbubble has a diameter from at least about 0.5 μm to at least about 5 μm.

When producing the targeted microbubbles, the contacting of the phospholipid-ligand conjugate, the phospholipid and the inert gas may occur in a specific device. In some embodiments, the device is a microfluidic device. In some embodiments, the device is a mechanical agitation device.

In another aspect of the invention, compositions comprising the phospholipid-ligand bioconjugates or the targeted microbubbles are provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1D. Characterization of affibodies (ABYs) with a C-terminal cysteine (cys) residue. (A) Schematic representation of ABY-fusion protein expression format and protein design with an N-terminal His-Tag followed by enterokinase cleavage (Asp-Asp-Asp-Asp-Lys) site, and affibody sequences. The ABY expresses a single C-terminal cysteine (Cys) following a pentaglycine (Gly-Gly-Gly-Gly-Gly; SEQ ID NO: 1) bridge. MALDI-TOF graph of AC12 ABY shows peaks representing a singly charged species (8.266 kDa) and a doubly charged species (4.1 kDa). (B) Comparison of two B7-H3-specific ABY_{cys} (AC2 and AC12) protein ligands resolved in of SDS-PAGE and stained by Coomassie blue. AC12 expression analysis exhibits higher purity when compared to the AC2. Ligand expression shows primarily a monomer protein band at ˜8 kDa size. (C) Flow cytometry-based binding comparison of two ABY ligands (1 μM) to the wild-type (MS1_{WT}) and B7-H3-expressing (MS1_{B7-H3}) endothelial cell lines. Biotinylated-AC12 shows higher target-binding specificity compared to the biotinylated-AC2 ligand as detected by streptavidin Alexa Fluor 647 (AF647) interaction. (D) Flow cytometry-based binding of biotinylated-ABY ligand (1 μM) to cell-surface B7-H3 isoforms expressed by human (MDA-MB-231) and murine (4T1) breast cancer cell lines. Anti-B7-H3 APC antibody staining was used as positive control, which shows expression levels of corresponding B7-H3 isoforms in these cell lines. AC12 shows superior binding affinity to human isoform while showing the binding strength to murine isoform of B7-H3 comparable to the AC2 ligand.

FIG. 2A-2C. Site-specific conjugation of ABY-cys with lipid-maleimide (Mal). (A) Coomassie blue staining (left) and fluorescence imaging (right) of SDS-PAGE gel confirming conjugation of the Mal derivative of Alexa Fluor 647 dye (˜1.2 kDa) with the reduced form (reactive free thiol group) of the AC12 ligand at 10:1 molar ratio. The unlabeled ABY band does not show fluorescence signal. (B) Coomassie blue staining of SDS-PAGE gel confirming conjugation of DSPE-PEG-Mal with TCEP reduced AC12 (10:1, 15:1, and 20:1 molar ratios) incubated at room temperature for 2 h. A strong band representing the conjugate (>11 kDa) formed above the AC12-cys monomer under these conditions. Staining also shows diffused bands of lipids (˜2.9 kDa) and a small proportion of ABY dimer (>17 kDa). (C) Coomassie blue staining of SDS-PAGE gel examining thiol-maleimide reaction specificity by conjugation of DSPE-PEG-Mal with thiol reduced or non-reduced forms of AC12 (20:1 molar ratio), and a control conjugation reaction with DSPE-PEG (no Mal functional group) and reduced form of AC12, incubated at 4° C. for 16 h. This figure indicates the reduced AC12 conjugated with DSPE-PEG-Mal but not with the control DSPE-PEG, while partial conjugation was achieved with the non-reduced AC12. Undesirable products, such as ABY aggregates (dimers) and its non-specific phospholipid conjugation (*) were observed in high proportions, especially with the non-reduced ABY form, under these reaction conditions.

FIG. 3A-3D. Production of targeted microbubbles (MBs) using a microfluidic system. (A) Left: MB formulation scheme representing phospholipid mixture, DPPC (95 mole %) and DSPE-PEG or DSPE-PEG-ABY bioconjugate (5 mole %). Right: Histograms showing mean MB size (mean diameter: ˜1.3 μm) and concentration (˜1.2×10⁹/mL) for control MBs without the bioconjugate (MB_{NT}; upper graph) and targeted MBs with the ABY bioconjugate (MB_{B7-H3}; lower graph), produced using a microfluidic system (Horizon Microbubble Maker, University of Leeds, U.K. (B) Histograms showing flow cytometry-based confirmation of His-Tag tagged ABY displayed on the targeted MB (MB_{B7-H3}) shell (produced using microfluidics) by anti-His-Tag-APC antibody staining. Background staining of anti-His-Tag APC is low for the control MB_{NT}. (C) Fluorescence microscopy-based signal confirmation of MB_{B7-H3} labeled with Cell Mask dye against phospholipid shell (green) and anti-His-Tag-APC antibody against ABY (red). The composite image shows overlap (yellow) between MBs and ABY signals. (D) Histograms showing the flow cytometry confirmation of His-Tag tagged ABY displayed on the MB_{B7-H3} produced by mechanical agitation method (VialMix).

FIG. 4A-4D. In vitro and in vivo binding validation of targeted microbubbles. (A) Flow cytometry assay was performed with DSPE-PEG (control) and DSPE-PEG-ABY using MS1_{WT} and MS1_{B7-H3} cells followed by anti-His-Tag-APC antibody staining. The DSPE-PEG-ABY shows strong and specific binding to the MS1_{B7-H3} cells. (B) Phase contrast microscopic images showing enhanced binding of MB_{B7-H3} (red circles) to a monolayer of MS1_{B7-H3} cells (upper panels) compared to the MS1_{WT} cells (lower panels). MB_{NT} binding was low for both types of cells. (C) Representative bar graph showing statistically significant (marked by ‘*’) counts of MB_{B7-H3} binding (red circles) to MS1_{B7-H3} cells compared to all other experimental groups. (D) Confocal microscopic images representing tissue immunofluorescence staining for vascular B7-H3 (red channel, Alexa Fluor 594) and intravenously injected MB_{B7-H3} (green channel) pre-labeled with fluorescent dye (Cell Mask Green), within the tumor tissues of a transgenic breast cancer mouse (MMTV-PyMT). Composite images confirm the localization of MB_{B7-H3} to tumor tissue expressing the vascular B7-H3 target. DAPI (blue) represents nuclear staining for cells.

FIG. 5A-5B Schematic illustration of the overall study plan with the preparation of B7-H3 targeted microbubbles using a microfluidic system. (A) Schematic workflow of the overall study plan showing the preparation and evaluation of pharmaceutical grade B7-H3 targeted microbubbles using affibodies (ABYs). (B) Pictorial representation showing the preparation of targeted microbubbles using a microfluidic device.

FIG. 6. His-Tag removal from ABY ligand to enable its clinical translation. SDS-PAGE analysis of 10 μg AC12 protein treated overnight with enterokinase (EK; 0.4 or 4 IU) to remove the N-terminal His-Tag from the ABY (˜8.3 kDa). EK (4 IU) completely cleaved (arrows) AC12 ABY (both monomer ˜6.9 kDa and dimer ˜13.8 kDa) expressed from plasmid with proteolytic recognition sequence (Asp-Asp-Asp-Asp-Lys) placed between the N-terminal His-Tag and the ABY sequences. EK proteolysis-associated protein bands were observed with the positive control protein but not with ABY expressed without the EK recognition site. High amounts of ABY dimer formation in the cleavage assay are observed due to overnight incubation of the non-reduced ABY form to allow optimal EK-mediated cleavage activity.

FIG. 7A-7B. MALDI-TOF analysis and purification of DSPE-PEG-ABY bioconjugate mixture. (A) MALDI-TOF analysis of solutions containing DSPE-PEG-Mal phospholipid (top) and AC12cys (middle) in separate solutions or in conjugation mixture (DSPE-PEG-ABY; bottom). m/z peak for the conjugation sample (11307 Da) confirms bioconjugation of AC12cys (Peak m/z: 8276 Da) with DSPE-PEG-Mal (peak m/z: 2926 Da). (B) SDS-PAGE analysis of the bioconjugation reaction mixture purified and concentrated through centrifugal membranes with ˜3 kDa molecular weight size cut-offs to remove small impurities before the use of bioconjugate mixture in targeted MB production. No sample loss is observed during this step. DSPE-PEG and AC12cys ABY samples are used for reference.

FIG. 8A-8B. Stability of DSPE-PEG-ABY bioconjugate under various storage conditions. (A) SDS-PAGE analysis of bioconjugate sample mixture consisting of AC12cys and DSPE-PEG-Mal stored in freeze-dried form at −20° C. for 3 weeks or in water at 4° C. for 2 weeks. No bioconjugate degradation is observed in both storage conditions, but a high molecular-weight band, suggesting non-specific aggregation (*), is observed in sample stored at 4° C. (B) SDS-PAGE analysis of bioconjugate mixture stored in Lamelli buffer (containing SDS and reactive primary amines from Tris) at 4° C. for 3 weeks. Partial degradation (*) of the bioconjugate product is observed under this sub-optimal storage condition.

FIG. 9. In vivo MBB7-H3 localization in normal tissues. Representative composite fluorescence images from tumor-adjacent normal tissue (left), liver (middle), and kidney (right) showing signals from dye labeled MBB7-H3 (green) and anti-B7-H3 Alexa Fluor 594 (red) immunostaining. Only liver shows diffuse signal for MBs suggesting their rapid hepatic clearance, while vascular B7-H3 is absent in all three tissue types. DAPI (blue) represents nuclear staining for cells.

FIG. 10A-10C. Measurement of MBs size prepared by the vial mixing and microfluidic methods. MBs were prepared by two (vialmix and microfluidic) approaches and compared the bubbles count and size using acoustic spectroscopy for particle size measurement. Panel A: MBs prepared by vial mix; panel B and panel C: Non-targeted and Targeted MBs prepared by Horizon microfluidic system. Acoustic spectroscopy clearly shown that MBs prepared by Horizon microfluidic system resulted with MBs of uniform and narrow size distribution compared to the vial mix based preparation.

### DETAILED DESCRIPTION OF THE EMBODIMENTS OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

By “inert gas” it is meant any gas that does not undergo chemical reactions under the conditions disclosed within the present disclosure. Inert gases comprise a range of different gases include those known as the noble gases.

By “mole percent” it is meant that the number of moles present is a specific percentage of the total moles of all substance within a given structure (i.e. a target microbubble).

The term “thiol-Michael addition reaction” as used herein is meant to indicate an organic reaction between a thiol and an ene compound with electron-withdrawing group. This reaction is sometimes referred to a “click reaction”. A thiol-Michael addition reaction may be represented by the following reaction:

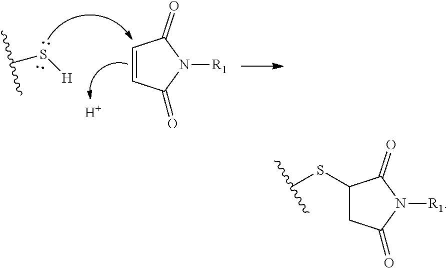

“PEG,” “polyethylene glycol” and “poly(ethylene glycol)” as used herein, are interchangeable. Typically, PEGS for use in accordance with the invention comprise the following structure: “—(OCH₂CH₂)n-” where (n) is 2 to 4000. As used herein, PEG also includes “—CH₂CH₂—O(CH₂CH₂O)ₙ—CH₂CH₂—” and “—(OCH₂CH₂), O—,” depending upon whether or not the terminal oxygens have been displaced. Throughout the specification and claims, it should be remembered that the term “PEG” includes structures having various terminal or “end capping” groups. The term “PEG” also means a polymer that contains a majority, that is to say, greater than 50%, of —OCH₂CH₂— or —CH₂CH₂O-repeating subunits. With respect to specific forms, the PEG can take any number of a variety of molecular weights, as well as structures or geometries such as “branched,” “linear,” “forked,” “multifunctional,” and the like.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, Ill, IV or V) or grade (G1, G2, G3, etc.) of neoplasia, tumor, or cancer, or a neoplasia, tumor, cancer or metastasis that is progressing, worsening, stabilized or in remission.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fᵥ molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

A “single-chain antibody,” “single chain variable fragment,” or “scFv” comprises an antibody heavy chain variable domain (VH) and a light-chain variable domain (VL) joined together by a flexible peptide linker. The peptide linker is typically 10-25 amino acids in length. Single-chain antibodies retain the antigen-binding properties of natural full-length antibodies, but are smaller than natural intact antibodies or Fab fragments because of the lack of an Fc domain.

B7-H3 (CD276) is a 316-amino acid (aa) type I transmembrane glycoprotein belonging to the immunoglobulin superfamily that contains a putative 28 aa signal peptide, a 217 aa extracellular region with one V-like and one C-like Ig domain, a transmembrane region and a 45 aa cytoplasmic domain. Its molecular weight is ˜45-66 kDa. As a result of exon duplication, the extracellular architecture of B7-H3 is characterized by a single IgV-IgC-like (2IgB7-H3) or IgV-IgC-IgV-IgC-like domain containing conserved cysteine residues. The predominant isoform in human tissues and cell lines is 4IgB7-H3 rather than 2IgB7-H3. The B7-H3 gene is located on chromosome 15 in humans and on chromosome 9 in mice. This gene consists of ten exons, among which exons 4 to 7 encode the extracellular IgV-IgC domains. B7-H3 is one of the most evolutionarily conserved B7 family members. In some embodiments, human B7-H3 is defined by the amino acid sequence:

(SEQ ID NO: 2)
MLRRRGSPGMGVHVGAALGALWFCLTGALEVQVPEDPVVALVGTDATLC
CSFSPEPGFSLAQLNLIWQLTDTKQLVHSFAEGQDQGSAYANRTALFPD
LLAQGNASLRLQRVRVADEGSFTCFVSIRDFGSAAVSLQVAAPYSKPSM
TLEPNKDLRPGDTVTITCSSYQGYPEAEVFWQDGQGVPLTGNVTTSQMA
NEQGLFDVHSILRVVLGANGTYSCLVRNPVLQQDAHSSVTITPQRSPTG
AVEVQVPEDPVVALVGTDATLRCSFSPEPGFSLAQLNLIWQLTDTKQLV
HSFTEGRDQGSAYANRTALFPDLLAQGNASLRLQRVRVADEGSFTCFVS
IRDFGSAAVSLQVAAPYSKPSMTLEPNKDLRPGDTVTITCSSYRGYPEA
EVFWQDGQGVPLTGNVTTSQMANEQGLFDVHSVLRVVLGANGTYSCLVR
NPVLQQDAHGSVTITGQPMTFPPEALWVTVGLSVCLIALLVALAFVCWR
KIKQSCEEENAGAEDQDGEGEGSKTALQPLKHSDSKEDDGQEIA

The term diabody as used herein refers to a bispecific antibody. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) and light-chain variable domain (VL) joined by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fᵥ (sFv) dimers has also been reported. See Gruber et al., J. Immunol., 152:5368 (1994). Alternatively, the antibodies can be “linear antibodies” as described in Zapata et al. Protein Eng. 8(10): 1057-1062 (1995). Briefly, these antibodies comprise a pair of tandem Fd segments (VH—CH1—VH—CH1) which form a pair of antigen binding regions. Linear antibodies can be bispecific or monospecific.

Affibodies are designed protein molecules developed on a scaffold, for example the three-helix bundle derived from the Z domain of staphylococcal protein A. By randomizing amino acids on two of the three helices, large libraries can be constructed, from which potent binders can be isolated by a variety of display method. Affibody molecules can be selected to a large variety of different proteins, and can be functionalized with genetic fusions to protein modules or by chemical conjugation to functional moieties, such as toxins, imaging agents, epitope tags, and the like.

Affibody proteins have very small size and hence favorable properties for diagnostic imaging because a molecular size below 10 kDa allows more rapid extravasation from blood vessels and penetration into tissue, allowing for rapid reach of tumor targets, and a short plasma half-life. Imaging is an important tool to identify, characterize and monitor tumors. Affibodies for imaging purposes may be conjugated, for example to chelating agents for complexation of radiometals, and imaged by single photon computed tomography (SPECT), positron emission tomography (PET), fluorescent probes in the ultraviolet, near infrared, etc.

Affibody molecules have the ability to bind protein targets with high affinity and selectivity. In contrast to antibodies that have Fc, however, they lack half-life extension and effector function modules. Therapeutic action can thus either be directly carried out by blocking ligand receptor interactions, or by functionalizing the Affibody molecules to have long half-lives and toxic payloads. For example a small engineered albumin-binding domain (ABD) has been genetically fused to affibodies, and they have been conjugated to therapeutic radionuclides, or protein toxins such as Pseudomonas exotoxin. Affibodies have also been engineered in different formats for tailored kinetic properties, including PEGylation, Fc-fusion or fusion to ABD for albumin binding.

Affibody molecules have been combinatorially fused with antibodies to form functional multispecific proteins called ‘AffiMabs’, for example which have a symmetric bi-valency and Fc sequence of common IgGs, with corresponding substantial half-life and stability in vivo and facile manufacturability.

Microbubbles are important contrast agents for diagnostic, theranostic, or therapeutic purposes, in that they can provide simultaneous and co-localized contrast for imaging and drug carrying and delivering capacity for targeted therapy. The imaging modality and therapeutic trigger is ultrasound, which is focused to microscale events distributed throughout the insonified vasculature. A gas core, e.g. air, perfluorobutane, etc. provides the mechanism for ultrasound backscatter. Gas bubbles of this size in aqueous media are unstable owing to surface tension effects, and require a stabilizing shell. The shell may be composed of surfactants, lipids, proteins, polymers, or a combination of these materials.

Lipid-coated microbubble formulations are commercially available and approved for clinical use, e.g. Definity (Lantheus Medical Imaging) and Sonovue® (Bracco Diagnostics). Phospholipids spontaneously self-assemble into a highly oriented monolayer at the air-water interface, forming around a newly entrained gas bubble. Lipid-coated microbubbles have exhibited favorable ultrasound characteristics, such as resonance with minimal damping and the ability to reseal around the gas core following fragmentation, and are easily functionalized for drug delivery, molecular imaging or other purposes by incorporating different lipid headgroup species or post-production bio-conjugation. Examples include phosphatidyl choline and lipopolymers.

Microbubbles have useful effects when they are insonified by ultrasound. At low acoustic pressures, an insonified microbubble produces a backscattered echo that can be used to detect and locate the microbubble. The microbubble can therefore be used as a contrast agent in ultrasound imaging. The echogenicity, or relative strength of the backscattered signal, is strongest near the microbubble resonance frequency. Bubbles of a few micrometers in diameter resonate at frequencies in the 1-10 MHz range which is the range of typical ultrasound clinical imaging scanners. Thus, microbubbles are highly echogenic to conventional ultrasound. Additionally, microbubbles scatter ultrasound nonlinearly. Imaging pulse sequences with modulated phase, frequency and amplitude can be used to separate the microbubble and tissue signals with high fidelity.

At higher acoustic pressures, the microbubble may become unstable during oscillation and fragment into daughter bubbles. Fragmentation is a useful means of eliminating the contrast agent signal within the transducer focus. Microbubble fragmentation is being employed to measure reperfusion in tumor and cardiac tissue and in ultrasound molecular imaging protocols.

At acoustic pressures just below the fragmentation threshold, a microbubble will undergo dissolution, e.g. for drug delivery. At high acoustic pressures and lower frequencies inertial cavitation occurs and can be exploited for drug delivery.

Commercialization of advanced ultrasound scanner technology and contrast agent detection methods (e.g., Siemens' Cadence Pulse Sequencing® mode) has made microbubble contrast agents highly effective in imaging.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

As used herein, a “therapeutically effective amount” refers to that amount of the therapeutic agent, e.g. a targeted microbubble conjugated to a therapeutic molecule, sufficient to prevent, treat or manage a disease or disorder. A therapeutically effective amount may refer to the amount of therapeutic agent sufficient to delay or minimize the onset of disease, e.g., delay or minimize the spread of cancer, or the amount effect to decrease or increase signaling from a receptor of interest. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “treating” is used to refer to treatment of a pre-existing condition. The treatment of ongoing disease, to stabilize or improve the clinical symptoms of the patient, is a particularly important benefit provided by the present invention. Evidence of therapeutic effect may be any diminution in the severity of disease, e.g. reduction of tumor size, decrease in residual disease, etc. The therapeutic effect can be measured in terms of clinical outcome or can be determined by immunological or biochemical tests. Patients for treatment may be mammals, e.g. primates, including humans, may be laboratory animals, e.g. rabbits, rats, mice, etc., particularly for evaluation of therapies, horses, dogs, cats, farm animals, etc.

As used herein, the terms “prevent”, “preventing” and “prevention” refer to the prevention of the recurrence or onset of one or more symptoms of a disorder in a subject as result of the administration of a prophylactic or therapeutic agent. In certain instances, prevention indicates inhibiting or delaying the onset of a disease or condition, in a patient identified as being at risk of developing the disease or condition.

As used herein, the terms “cancer” (or “cancerous”), “hyperproliferative,” and “neoplastic” to refer to cells having the capacity for autonomous growth (e.g., an abnormal state or condition characterized by rapidly proliferating cell growth). Hyperproliferative and neoplastic disease states may be categorized as pathologic (e.g., characterizing or constituting a disease state), or they may be categorized as non-pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

### Methods of Producing Phospholipid-Ligand Bioconjugates

In one aspect, this application is directed to methods for producing phospholipid-ligand bioconjugates. The methods comprise contacting a phospholipid polymer comprising a maleimide-containing functional group with a ligand comprising a C terminal cysteine residue.

In some embodiments, the method of producing the phospholipid-ligand bioconjugate comprises heating the phospholipid polymer prior to the contacting step. The phospholipid polymer may be heated to a range of different temperatures. For instance, the phospholipid polymer may be heated to at least about 50° C., from at least about 50° C. to at least about 55° C., from at least about 55° C. to at least about 60° C., from at least about 60° C. to at least about 65° C., from at least about 65° C. to at least about 70° C., from at least about 70° C. to at least about 75° C., from at least about 75° C. to at least about 80° C., or greater than about 80° C. The phospholipid may be heated for a specific period of time. For example, the specific amount of time may be at least about 2 hours, at least about 2.5 hours, at least about 3 hours, at least about 3.5 hours, at least about 4 hours, at least about 4.5 hours, at least about 5 hours, or greater than 5 hours.

In embodiments where the phospholipid polymer is heated, following heating the phospholipid polymer may be reduced to room temperature for a specific period of time. For instance, the phospholipid polymer may be reduced to room temperature for at least about 0.5 hours, at least about 1 hours, at least about 1.5 hours, at least about 2 hours, at least about 2.5 hours, at least about 3 hours, at least about 3.5 hours, at least about 4 hours, or greater than 4 hours.

Phospholipid-ligand bioconjugates of the present disclosure may be comprised of a range of different phospholipid polymers and ligands. Phospholipid polymers for use in the present disclosure include any phospholipid polymer capable of being modified with a maleimide or its derivative-containing functional group. Phospholipid polymers that find use in the present disclosure include, without limitation, DSPE-PEG(2000). 1,2-distearyol-sn-glycero-3-phosphoethanolamine-N-[amino(polyethylene glycol) 2000] (DSPE-PEG2000-A), 1,2-distearyol-sn-glycero-3-phosphoethanolamine-N-[biotinyl (polyethylene glycol)2000] (DSPE-PEG2000-B) and 1,2-distearyol-sn-glycero-3-phosphoethanolamine-N-[methoxy(polyethylene glycol) 5000](DSPE-PEG5000), etc.

Ligands for use in the present disclosure include any ligand able to specifically bind to a protein, lipid, or small molecule present on the surface of a cell such as a cancer cell. Ligands that find use in the present disclosure include, without limitation, affibodies, single chain variable fragments, VHH antibodies, single domain antibodies, diabodies, etc. The ligands may bind to any protein, lipid, or small molecule deemed useful, particularly with respect to ultrasound-based molecular imaging applications. Proteins of particular interest include proteins that are specifically expressed on cancer cells (e.g. B7-H3). Non-limiting examples of other potential proteins or tumor specific antigens include CD19, CAIX, CEA, CD5, CD7, CDIO, CD20, CD22, CD30, CD33, CD34, CD38, CD41, CD44, CD49f, CD56, CD74, CD90, CD123, CD133, CD138, a cytomegalovirus (CMV) infected cell antigen, EGP-2, EGP-40, EpCAM, erb-B2,3,4, FSP, Fetal acetylcholine receptor, folate receptor-a, GD2, GD3, HER-2, hTERT, IL-13R-.alpha.2, K-light chain, KDR, LeY, LI cell adhesion molecule, MAGE-AI, Mesothelin, Muc-1, Muc-16, NKG2D ligands, NY-ESO-1, oncofetal antigen (h5T4), PSCA, PSMA, RORI, TAG-72, VEGF-R2, WT-1, and the like. In some embodiments, the ligands of the present disclosure are versions, e.g., a scFv version, an affibody version, a VHH antibody, a single domain antibody version, a diabody version, etc., of an antibody approved by the United States Food and Drug Administration and/or the European Medicines Agency (EMA) for use as a therapeutic antibody. Non-limiting examples of ligands that may be versions of Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof.

The cells that the ligands are targeted to may be any cell of interest. Of particular interest are endothelial and cancer cells associated with solid tumors. Cancer cells of interest include, without limitation, cells from adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Kaposi's sarcoma, kidney cancer, laryngeal and hypopharyngeal cancer, liver cancer, lung cancer, lung carcinoid tumors, male breast cancer, malignant mesothelioma, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, or teratocarcinoma.

In some embodiments, the ligand is specific to B7-H3. In some embodiments, the ligand is selected from the group consisting of AC2, AC9, AC12, and AC16. In some embodiments, the ligand is AC2. When the ligand is AC2, the ligand may have an amino acid sequence according to AEAKYAKEKIFAVGEIYWLPNLTHGQIMAFIAALNDDPSQSSELLSEAKKLNDSQAPK (SEQ ID NO: 3). In some embodiments, the ligand is AC9. When the ligand is AC9, the ligand may have an amino acid sequence according to
AEAKYAKEKIIALSEIIWLPNLTHGQIMAFIAALNDDPSQSSELLSEAKKLNDSQAPK (SEQ ID NO: 4). In some embodiments, the ligand is AC12. When the ligand is AC12, the ligand may have an amino acid sequence according to
AEAKYAKEKIAALSEIIWLPNLTHGQIMAFIAALNDDPSQSSELLSEAKKLNDSQAPK (SEQ ID NO: 5). In some embodiments, the ligand is AC16. When the ligand is AC16, the ligand may have an amino acid sequence according to
AEAKYAKEKVHALSEIIWLPNLTHGQIMAFIAALNDDPSQSSELLSEAKKLNDSQAPK (SEQ ID NO: 6). Ligands specific to B7-H3 have been described in the art by, for example, Stern et al. (ACS Comb. Sci. 2019, 21, 207-222) which is specifically incorporated by reference.

All ligands of the present disclosure comprise a C terminal cysteine residue. In some embodiments, the C terminal cysteine is preceded by a linker. In some embodiments, the linker comprises 2 to 10 amino acids. In some embodiments, the peptide linker comprises 2, 3, 4, 5, 6, 7, 8, 9, 10, 11, 12, 13, 14, 15, 16, 17, 18, 19, 20 or greater than 20 amino acids. In some embodiments, the peptide linker is between 1 to 5, 1 to 10, 1 to 15, 1 to 20, 5 to 10, 5 to 15 or 5 to 20 amino acids in length. Exemplary linkers include linear peptides having at least two amino acid residues such as Gly-Gly, Gly-Ala-Gly, Gly-Pro-Ala, Gly-Gly-Gly-Gly-Ser (SEQ ID NO: 7). Suitable linear peptides include poly glycine, polyserine, polyproline, polyalanine and oligopeptides consisting of alanyl and/or serinyl and/or prolinyl and/or glycyl amino acid residues. In some embodiments, the peptide linker comprises the amino acid sequence selected from the group consisting of Gly₉ (SEQ ID NO: 8), Glu₉ (SEQ ID NO: 9), Ser₉ (SEQ ID NO: 10), Gly₅-Cys-Pro₂-Cys (SEQ ID NO: 11), (Gly₄-Ser)₃ (SEQ ID NO: 12), Ser-Cys-Val-Pro-Leu-Met-Arg-Cys-Gly-Gly-Cys-Cys-Asn (SEQ ID NO: 13), Pro-Ser-Cys-Val-Pro-Leu-Met-Arg-Cys-Gly-Gly-Cys-Cys-Asn (SEQ ID NO: 14), Gly-Asp-Leu-Ile-Tyr-Arg-Asn-Gln-Lys (SEQ ID NO: 15), and Gly₉-Pro-Ser-Cys-Val-Pro-Leu-Met-Arg-Cys-Gly-Gly-Cys-Cys-Asn (SEQ ID NO: 16). In some embodiments, the linker is a pentaglycine bridge (i.e. Gly₅; SEQ ID NO: 1).

The phospholipid polymers of the present disclosure may be contacted with the ligand under specific conditions. The specific conditions may include contacting the phospholipid with the ligand is a particular ratio or performing the contacting at a specific pH. A range of different phospholipid to ligand ratios are appropriate including, without limitation, 1 to 1, 2 to 1, 3 to 1, 4 to 1, 5 to 1, 6 to 1, 7 to 1, 8 to 1, 9 to 1, 10 to 1, 11 to 1, 12 to 1, 13 to 1, 14 to 1, 15 to 1, 16 to 1, 17 to 1, 18 to 1, 19 to 1, 20 to 1, 21 to 1, 22 to 1, 23 to 1, 24 to 1, 25 to 1, 26 to 1, 27 to 1, 28 to 1, 29 to 1, 30 to 1, etc. In a preferred embodiment, the phospholipid is contacted with the ligand in a 20 to 1 ratio (20 phospholipid to 1 ligand). A range of different pH is appropriate including, without limitation, 6 pH, 6.5 pH, 7 pH, 7.5 pH, 8 pH, etc. In some embodiments, the contacting step is performed at a neutral pH (i.e. 7 pH).

The phospholipid-ligand bioconjugates may have particular physical characteristics or features. For example, the phospholipid-ligand bioconjugates may have prolonged stability at low temperatures or retain binding activity after a number of freeze thaw cycles or lyophilization. The prolonged stability relates to a lack of degradation of the phospholipid-ligand bioconjugate when at low temperatures. The prolonged stability may be at least about 3 weeks, at least about 4 weeks, at least about 5 weeks, at least about 6 weeks, at least about 7 weeks, at least about 8 weeks, at least about 9 weeks, at least about 10 weeks, or greater than 10 weeks. The phospholipid-ligand bioconjugate may retain binding activity after a range of different freeze thaw cycles. For instance, phospholipid-ligand bioconjugate may retain binding activity after 3, 4, 5, 6, 7, 8, 9, 10, 11, 12, 13, 14, 15, 16, 17, 18, 19, 20 or great than 20 freeze thaw cycles.

### Methods of Producing Target Microbubbles

In one aspect, this application is directed to methods for producing targeted microbubbles. The methods comprise contacting a micelle comprising a phospholipid-ligand bioconjugate with a phospholipid liposome and an inert gas. In some embodiments, two or more different micelles comprising phospholipid-ligand bioconjugates are contacted with the phospholipid liposome and the inert gas. For instance, there may be a first phospholipid-ligand bioconjugate that has a ligand that targets a first protein and a second phospholipid-ligand bioconjugate that has a ligand that targets a second protein that is different from the first protein.

As used herein, “microbubbles” refer to micron-sized contrast agents composed of a shell and a gas core, as is well known to those of skill in the art. The shell may be formed from any suitable material, including but not limited to albumin, polysaccharides (such as galactose), lipids (such as phospholipids), polymers, and combinations thereof. Any suitable gas core can be used in the microbubbles of the invention, including but not limited to air, perfluorocarbons (octafluoropropane, perfluorobutane), sulfur hexafluoride, nitrogen, etc. The gas core determines the echogenecity of the microbubble. When gas bubbles are caught in an ultrasound frequency field, they compress, oscillate, and reflect a characteristic echo, this generates the strong and unique sonogram in contrast-enhanced ultrasound. Gas cores can be composed of air, or heavy gases like octafluoropropane, perfluorocarbon, sulfur hexafluoride or nitrogen. Heavy gases are less water-soluble so they are less likely to leak out from the microbubble to impair echogenecity. Inert gases of the invention include, without limitation, perfluorobutane, octafluoropropane, perfluorocarbon, sulfur hexafluoride, helium, neon, argon, krypton, xenon, radon, oganesson, etc.

The production of targeted microbubbles may involve the use of a number of different phospholipid liposomes. Phospholipid liposomes that find use in the present disclosure include without limitation, DPPC, DSPC, DPPA, DPPG, etc. Phospholipid liposomes may also have specific physical characteristics such as having a particular diameter. For instance, the diameter of the phospholipid liposomes may have a diameter from at least about 50 nm to at least about 60 nm, at least about 60 nm to at least about 70 nm, at least about 70 nm to at least about 80 nm, at least about 80 nm to at least about 90 nm, at least about 90 nm to at least about 100 nm, at least about 100 nm to at least about 110 nm, at least about 110 nm to at least about 120 nm, at least about 120 nm to at least about 130 nm, at least about 130 nm to at least about 140 nm, at least about 140 nm to at least about 150 nm, or greater than 150 nm in diameter.

The targeted microbubbles of the present disclosure may comprise specific amounts of phospholipid-ligand conjugates and phospholipids. The specific amount of phospholipid-ligand conjugates and phospholipids may be any specific amount to achieve a desired result, e.g. to achieve a specific diameter of the target microbubble, achieve a specific amount of uniformity between the targeted microbubbles produced, etc. In some embodiments, the targeted microbubbles comprise 1-10 mole percent phospholipid-ligand bioconjugate. The amount of the phospholipid-ligand bioconjugate may be any intervening amount. For instance, the targeted microbubbles may comprise at least about 1 mole percent, at least about 2 mole percent, at least about 3 mole percent, at least about 4 mole percent, at least about 5 mole percent, at least about 6 mole percent, at least about 7 mole percent, at least about 8 mole percent, at least about 9 mole percent or at least about 10 mole percent phospholipid-ligand bioconjugate. In some embodiments, the targeted the microbubble comprises 90-99 mole percent phospholipid. The amount of the phospholipid may be any intervening amount. For instance, the targeted microbubbles may comprise at least about 91 mole percent, at least about 92 mole percent, at least about 93 mole percent, at least about 94 mole percent, at least about 95 mole percent, at least about 96 mole percent, at least about 97 mole percent, at least about 98 mole percent, at least about 99 mole percent phospholipid.

The targeted microbubbles may also have specific size characteristics. In some embodiments, the targeted microbubble has an average diameter from at least about 0.5 μm to at least about 10 μm. The targeted microbubble may have any intervening average diameter. For instance, the targeted microbubble of at least about 0.5 μm, at least about 1 μm, at least about 1.5 μm, at least about 2 μm, at least about 2.5 μm, at least about 3 μm, at least about 3.5 μm, at least about 4 μm, at least about 4.5 μm, at least about 5 μm, at least about 5.5 μm, at least about 6 μm, at least about 6.5 μm, at least about 7 μm, at least about 7.5 μm, at least about 8 μm, at least about 8.5 μm, at least about 9 μm, at least about 9.5 μm, or at least about 10 μm in average diameter.

When producing the targeted microbubbles, the contacting of the phospholipid-ligand conjugate, the phospholipid and the inert gas may occur in a specific device. In some embodiments, the device is a microfluidic device. In some embodiments, the device is a mechanical agitation device. The specific device used may alter the characteristics of the targeted microbubbles produced. For instance, microbubbles produced by the microfluidic device may have more uniform diameter.

In one aspect, the application is directed to methods for producing targeted microbubbles conjugated to therapeutic molecules. The methods comprise contacting a first micelle comprising a phospholipid-ligand bioconjugate, such as those described above, with a second micelle comprising a phospholipid-therapeutic agent bioconjugate, a phospholipid liposome, and an inert gas. The targeted microbubbles conjugated to therapeutic molecules comprise a phospholipid-ligand bioconjugate that targets a protein, lipid, or small molecule present on the surface of a cell and a phospholipid-therapeutic agent bioconjugate that delivers the therapeutic agent to a target cell such as a cancer cell. The targeted microbubbles conjugated to therapeutic agents may be produced using the methods described above.

Therapeutic moleucles or agents for use in the present disclosure include any therapeutic agent that has a therapeutic effect on a target cell such as a cancer cell. Therapeutic agents that find use in the present disclosure include, without limitation, chemotherapeutic agents, toxins, radioactive isotopes, kinase inhibitors, immunomodulators, hormone blockers, etc. In some embodiments, the therapeutic agent is conjugated to the phospholipid in a manner such as those described above, i.e., with a terminal cysteine or a maleimide functional group.

The phospholipid-therapeutic agent bioconjugate of the present disclosure may comprise a chemotherapeutic agent. Chemotherapeutic agents include, without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon

The phospholipid-therapeutic agent bioconjugate of the present disclosure may comprise a toxin. Toxins that find use in the present disclosure include toxins of animal, plant or microbial origin. Exemplary toxins include Pseudomonas exotoxin, ricin, abrin, Staphylococcal enterotoxin-A, pokeweed antiviral protein, gelonin, diphtheria toxin, and Pseudomonas endotoxin.

The phospholipid-therapeutic agent bioconjugate of the present disclosure may comprise a radioactive isotopes. Particularly useful therapeutic radionuclides include, but are not limited to ¹¹¹In, ¹⁷⁷Lu, ²¹²Bi, ²¹³Bi, ²¹¹At, ⁶²Cu, ⁶⁴Cu, ⁶⁷Cu, 90Y, ¹²³I, ¹²⁵I, ¹³¹I, ³²P, ³³P, ⁷⁷Br, ⁴⁷Sc, ¹¹¹Ag, ⁶⁷Ga, ¹⁴²Pr, ¹⁵³Sm, 161Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh ¹⁰⁹Pd, ¹⁴³Pr, ¹⁴⁹Pm, ¹⁶⁹Er, ¹⁹⁴Ir, ¹⁹⁸Au, ¹⁹⁹Au, ¹¹C, ¹³N, ¹⁵O, ⁷⁵Br, ¹⁹⁸Au, ²²⁴Ac, 1261, 1331, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ¹²²mTe, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵¹Cr, ⁵⁹Fe, ⁷⁵Se, ²⁰¹TI, ²²⁵Ac, ⁷⁶Br, ¹⁶⁹Yb, and the like.

The phospholipid-therapeutic agent bioconjugate of the present disclosure may comprise a kinase inhibitor. The kinase inhibitor includes, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

The phospholipid-therapeutic agent bioconjugate of the present disclosure may comprise an immunomodulator. The immunomodulator may include a cytokine, a lymphokine, a monokine, a stem cell growth factor, a lymphotoxin (LT), a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), such as TGF-α or TGF-β, insulin-like growth factor (IGF), erythropoietin, thrombopoietin, a tumor necrosis factor (TNF) such as TNF-α or TNF-β, a mullerian-inhibiting substance, mouse gonadotropin-associated peptide, inhibin, activin, vascular endothelial growth factor, integrin, granulocyte-colony stimulating factor (G-CSF), granulocyte macrophage-colony stimulating factor (GM-CSF), an interferon such as interferon-α, interferon-β, or interferon-γ, S1 factor, an interleukin (IL) such as IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 or IL-25, LIF, kit-ligand, FLT-3, angiostatin, thrombospondin, endostatin, and LT.

The phospholipid-therapeutic agent bioconjugate of the present disclosure may comprise a hormone blocker. The hormone blocker may be a drug that blocks ER receptors (e.g. tamoxifen) or a drug that blocks the production of estrogen, such as an aromatase inhibitor (e.g. anastrozole, or letrozole).

### Methods of Treating Cancer

In another aspect of the invention, methods are provided for treating cancer, the method comprising contacting a cancer cell with an effective amount of a targeted microbubble conjugated to a therapeutic molecule such as those described above. In some embodiments, the targeted microbubble comprises a phospholipid-ligand bioconjugate comprising a ligand that specifically binds to a protein on the surface of the cancer cell.

The ligand may be any of the ligands described above. The ligand specifically targets a protein that is expressed on the surface of a cancer cell. Non-limiting examples of proteins or tumor specific antigens include B7-H3, CD19, CAIX, CEA, CD5, CD7, CDIO, CD20, CD22, CD30, CD33, CD34, CD38, CD41, CD44, CD49f, CD56, CD74, CD90, CD123, CD133, CD138, a cytomegalovirus (CMV) infected cell antigen, EGP-2, EGP-40, EpCAM, erb-B2,3,4, FSP, Fetal acetylcholine receptor, folate receptor-a, GD2, GD3, HER-2, hTERT, IL-13R-.alpha.2, K-light chain, KDR, LeY, LI cell adhesion molecule, MAGE-AI, Mesothelin, Muc-1, Muc-16, NKG2D ligands, NY-ESO-1, oncofetal antigen (h5T4), PSCA, PSMA, RORI, TAG-72, VEGF-R2, WT-1, and the like.

The cells that the ligands are targeted to may be any cancer cell of interest. Of particular interest are endothelial and cancer cells associated with solid tumors. Cancer cells of interest include, without limitation, cells from adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Kaposi's sarcoma, kidney cancer, laryngeal and hypopharyngeal cancer, liver cancer, lung cancer, lung carcinoid tumors, male breast cancer, malignant mesothelioma, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, or teratocarcinoma.

The therapeutic agent may be any of the therapeutic agents described above. In some embodiments, the therapeutic agent is selected from the group of a chemotherapeutic agent, a toxin, a radioactive isotope, a kinase inhibitor, an immunomodulator, or a hormone blocker.

Effective amounts of the targeted microbubble conjugated to a therapeutic molecule of the present invention for the treatment of cancer, vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage of the targeted microbubble conjugated to a therapeutic molecule may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Such dosage forms encompass physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The polypeptide will typically be formulated in such vehicles at a concentration of about 0.1 μg/ml to 100 μg/ml.

The compositions, e.g., a composition comprising the targeted microbubble or the targeted microbubble conjugated to a therapeutic molecule, and method of the present invention may be combined with additional therapeutic agents. For example, when the disease, disorder or condition to be treated is a neoplastic disease (e.g. cancer) the methods of the present in invention may be combined with conventional chemotherapeutic agents or other biological anti-cancer drugs such as checkpoint inhibitors (e.g. PD1 or PDL1 inhibitors) or therapeutic monoclonal antibodies (e.g Avastin, Herceptin).

Examples of chemical agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination may include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade), Jakafi (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

Examples of biological agents identified in the art as useful in the treatment of neoplastic disease, include without limitation, cytokines or cytokine antagonists such as IL-12, INFα, or anti-epidermal growth factor receptor, radiotherapy, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-β1a (Avonex®), and interferon-31b (Betaseron®) as well as combinations of one or more of the foregoing as practiced in known chemotherapeutic treatment regimens readily appreciated by the skilled clinician in the art.

Tumor specific monoclonal antibodies that can be administered in combination with an anti-CD93 ABD polypeptide or engineered cell may include, without limitation, Rituximab (marketed as MabThera or Rituxan), Alemtuzumab, Panitumumab, Ipilimumab (Yervoy), etc.

In some embodiments the compositions and methods of the present invention may be combined with immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda® MK-3475, lambrolizumab, commercially available from Merck and Company, Kenilworth NJ), and atezolizumab (Tecentriq®, Genentech/Roche, South San Francisco CA). Additional examples of PD1 inhibitory antibodies include but are not limited to durvalumab (MED14736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8(42): 72167-72181).

### Compositions

In another aspect of the invention, compositions comprising the phospholipid-ligand bioconjugates, the targeted microbubbles, or the targeted microbubbles conjugated to therapeutic agents are provided. The composition may further comprise a pharmaceutically acceptable excipient.

A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the pharmaceutical composition is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the pharmaceutical composition may further include a preservative. Suitable preservatives include, but are not limited to, a benzyl alcohol, phenol, chlorobutanol, benzalkonium chloride, and the like. In many cases, the formulation is stored at about 4° C. Pharmaceutical compositions may also be lyophilized, in which case they generally include cryoprotectants such as sucrose, trehalose, lactose, maltose, mannitol, and the like. Lyophilized formulations can be stored over extended periods of time, even at ambient temperatures.

The subject pharmaceutical composition can be administered orally, subcutaneously, intramuscularly, parenterally, or other route, including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The pharmaceutical composition may be administered in a unit dosage form and may be prepared by any methods well known in the art. Such methods include combining the targeted microbubble with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

The features and advantages of the present invention will be more clearly understood by reference to the following examples, which are not to be construed as limiting the invention.

### EXPERIMENTAL

### Example 1

### Introduction

Phospholipids-based microbubbles (MBs) serve as blood pool contrast agents and are used in conjunction with contrast-enhanced ultrasound imaging for diagnostic applications in cardiovascular and cancer imaging [1]. Molecularly targeted contrast agents that bind to receptors or biomarkers expressed in vascular targets in vivo are desirable due to their ability to enhance the specificity of disease diagnosis. To facilitate this process, MBs need to be conjugated with specific antibodies or engineered small protein ligands that can direct the MBs to bind to desired molecular targets in the blood vessels. Typically, the ligands are attached to the bubbles at the liquid interface of the phospholipid-monolayer shell by chemical conjugation methods. For example, KDR-targeted phospholipid MBs are designed to target tissue-specific pathological angiogenesis and have shown promising results in clinical trials [2,3]. Currently, there are no FDA-approved targeted MBs (TMBs) available for ultrasound molecular imaging. Moreover, the FDA-approved non-targeted MBs are produced by mechanical agitation method, which produce MBs of broader size distribution that can cause heterogeneous acoustic response with variabilities in ultrasound imaging sensitivity [4].

Developing a reproducible methodology to cater the synthesis of TMBs of uniform sizes with enhanced acoustic properties can improve the imaging sensitivity while facilitating the on-demand preparation of TMBs for ultrasound molecular imaging in clinical settings. Microfluidic-based processing methods offer better control over MB size dispersity for molecular imaging applications as well as improve the production efficiency and reproducibility [5,6]. Microfluidic devices produce MBs by a variety of pinch-off mechanisms with precisely controlled flow of gas and liquid streams. Chips fabricated with specific microfluidic channel geometry can consistently maintain production uniformity of MBs compared to those generated using sonication and amalgamation techniques [5]. Microfluidics-based devices can meet the requirements for clinically translatable TMB synthesis by providing an optimal platform technology.

Lipid-shell MB formulations consist of PEGylated phospholipids with functional groups (biotin/streptavidin, NHS esters, maleimide) that allow for the conjugation of antibodies or proteins to the MB shell. Surface-functionalization of proteins is often performed in pre-formed MBs with highly efficient chemical conjugation methods (streptavidin-biotin [7], NHS ester-amine [8], thiol-maleimide [9-12]); however, the MB labeling is not fully controlled in this approach, resulting in batch-to-batch variations. Further, ligand labeling in pre-formed MBs can affect the stability of MBs, and it is not a feasible strategy for clinical practice because technologists need readily injectable molecular contrast agents for patient imaging. Random chemical conjugations between phospholipids and antibodies, or proteins with multiple reactive side chains (e.g., lysine amino acids) can reduce the overall biological activity of the conjugated ligands [13]. To produce uniformly labeled TMBs for clinical use, phospholipid-ligand bioconjugates can be prepared prior to their integration into MBs in a controlled manner. Stable bioconjugate preparation using clinically applicable site-specific covalent bonding strategies between proteins and lipids can further enhance uniform MB labeling with targeting ligands [14]. MBs formulated with the incorporation of lipo-peptide bioconjugates binding to the KDR receptors have previously been applied with covalent amine conjugation chemistry [15]. Unlike larger proteins such as antibodies, engineered small protein ligands can be easily modified to promote site-specific bioconjugation reactions that are scalable and safer for therapeutic and imaging applications [16,17]. For example, small proteins can be modified to display a terminal cysteine residue, which upon reduction forms a stoichiometrically controlled and stable thioether bond to molecules with maleimide functional groups [18]. The site-specific covalent bonding methods could preserve ligand functionality while reducing variabilities in bioconjugation reactions, enhancing targeted MBs performance, and reproducibility.

Recent developments in microfluidic-based processing methods offer better control over the size distribution of the MBs. In addition, this tool has also shown reproducible production efficiency and size distribution [5,6]. Microfluidic devices produce MBs by a pinch-off mechanism through tapered flow channels with precisely controlled flow of gas and liquid streams. The microfluidic device used here can consistently maintain uniform production of desired MBs traits compared to those generated using sonication/amalgamation techniques [5]. Peyman et al. reported that MBs produced by a flow-focusing microfluidic device have higher ultrasound scattering properties over a wide range of frequencies compared to those formed by mechanical agitation methods [19]. In addition, microfluidic techniques can be applied to produce suspensions of uniformly sized MBs with contrast properties [20] tailored to specific ultrasound frequencies for improved imaging [21]. For contrast-enhanced molecular imaging applications, labeling consistency of ligands on surface of the MB shell is important for the optimal target-binding performance of targeted MBs. Furthermore, ligand labeling distribution can be enhanced upon preferential use of phospholipid formulations and specific lipid handling methods prior to MB production [22,23]. Microfluidic devices allow precise control over these production parameters to achieve standardized methodologies of targeted MB synthesis.

Herein, a clinically translatable synthesis of TMBs is presented, with an optimized workflow using a microfluidic system to directly incorporate targeting ligand or bioconjugates. In this TMB, the targeting moiety is an engineered affibody (ABY) protein selective against an angiogenic biomarker, the B7-H3 (CD276). B7-H3 is a type I transmembrane protein ligand with 316 amino acids (45-66 kDa) and belongs to a family of immune checkpoint molecules [24]. Several studies have shown that B7-H3 plays a role either as a co-stimulatory or as a co-inhibitory in T cell-mediated adaptive immunity [25]. However, when compared to tumor tissue, the level of expression is much lower in most other normal organs and tissues. In addition to tumors, it is predominantly expressed on the surface of T and B cells [24,26]. It has been reported that several cancer types express B7-H3 at higher level [20,27] which include prostate cancer [28,29], renal cell carcinoma [30], ovarian cancer [31], glioblastoma [32], osteosarcoma [33], pancreatic cancer [34], neuroblastoma [35], diffuse intrinsic pontine glioma, and mesothelioma [36, 37].

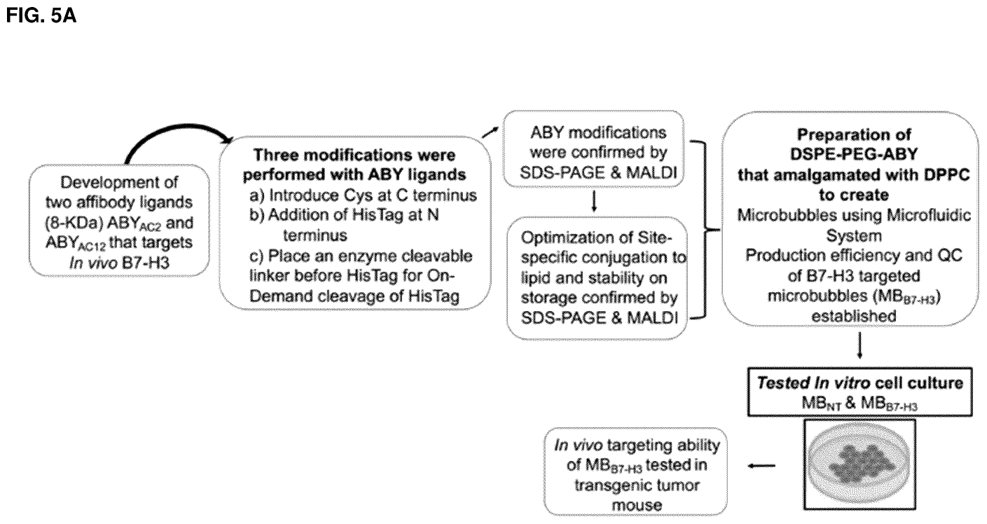

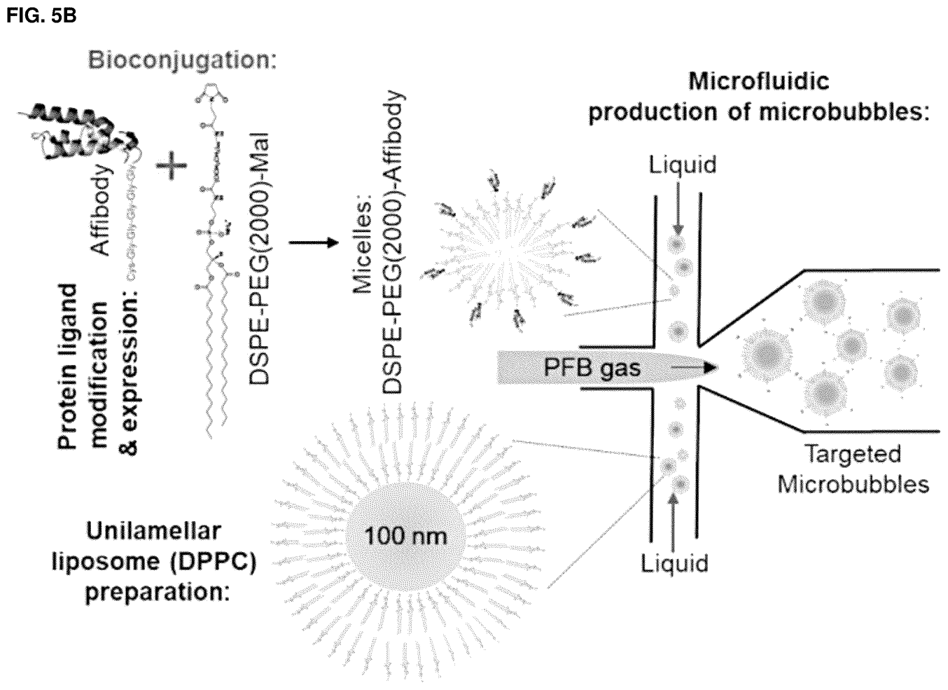

The ABY is engineered to express a cysteine residue at the C-terminus for a site-specific conjugation with phospholipids (DSPE-PEG-maleimide; 2 kDa) with an optimized thiol-maleimide chemical reaction to form stable bioconjugates and to be stable at physiological temperatures and pH. This novel TMB was characterized in terms of their size distribution, concentration, ligand display, and target-binding performance in vitro and in vivo. The workflow consistently results in uniform MB size, yield, and binding activity to its cellular target, B7-H3, expressed by vascular endothelial cells (FIG. 5).

### Results

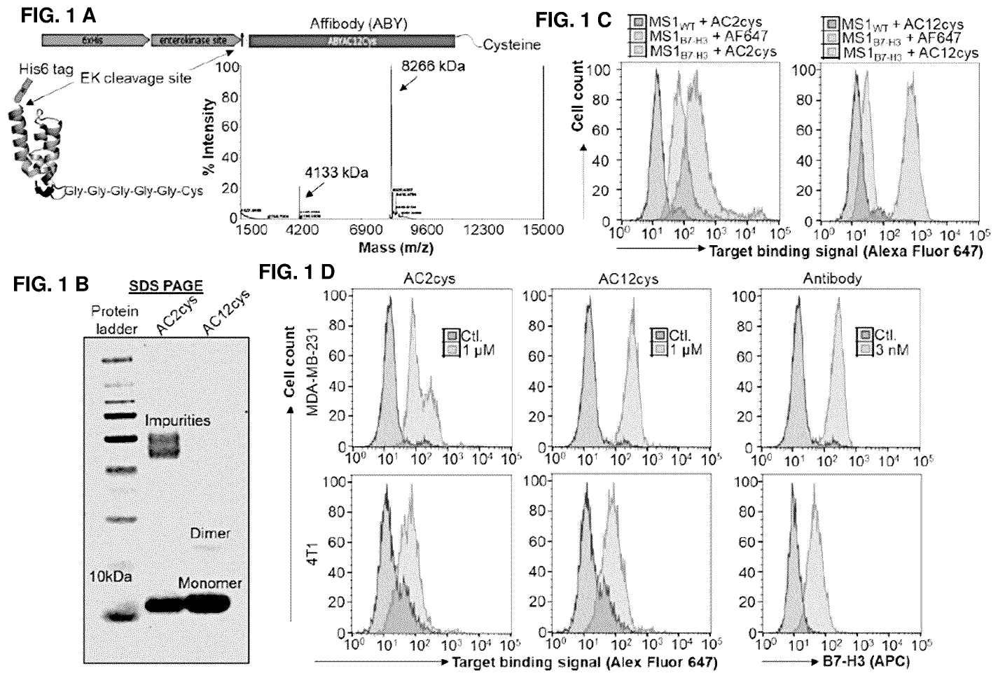

Expression and Target Binding Assessments of ABY Ligands after Site-Selective Protein Modifications. After successful engineering, the molecular weight of both the binders was tested by MALDI-TOF (FIG. 1A) and binding affinities to human/mouse B7-H3. Binding affinities for ABYAC2 and ABYAC12, which showed Kd of 310_100 nM and 0.9_0.6 nM, respectively. Similarly, purities of both ABYAC2 and ABYACI2 protein were assessed by the SDS-PAGE (FIG. 1B). The result shows one major band corresponding to ˜9 kDa for AC12 (lane #3) compared to AC2 (lane #2), which shows an expected band at ˜9 kDa and an additional high molecular weight band that could be possibly due to some aggregates. Further, the binding efficiency of ABYAC2 and ABYAC12 to B7-H3 was tested in MS1 mouse endothelial cells engineered to overexpress human B7-H3 (MS1 hB7-H3) by flow cytometry analysis. The result shows greater binding affinity by both engineered ABYs to endothelial cell line expressing B7-H3 compared to control MS1ctl cells (FIG. 1C). ABYAC12 showed low non-specific binding to MS1WT cells compared to ABYAC2, suggesting its higher binding specificity to B7-H3.

Preclinical characterization of ligands is important before their selections for clinical studies. Hence, the binding efficiency of ABY ligand to MDA-MB-231 human breast cancer cells expressing 4 lg B7-H3 isoform was tested, and 4T1 murine mammary cancer cells expressing 2 lg B7-H3 isoform. Both of these cell lines were confirmed for their corresponding B7-H3 isoforms expression by anti-B7-H3 antibody staining based flow cytometry assay (FIG. 1D). Interestingly, the binding efficiency of both ABYAC2 and ABYACI2 to 4T1 cells expressing murine B7-H3 was similar; however, the binding efficiency of ABYAC12 to human B7-H3 expressed on MDA-MB-231 cells was significantly higher compared to ABYAC2. These results are consistent with the previous findings that the engineering of ABYAC12 variant by affinity maturation of ABYAC2 against human B7-H3 resulted with enhanced target specificity, thermal stability, and refolding ability of the engineered ABYACI2 ligand [38].

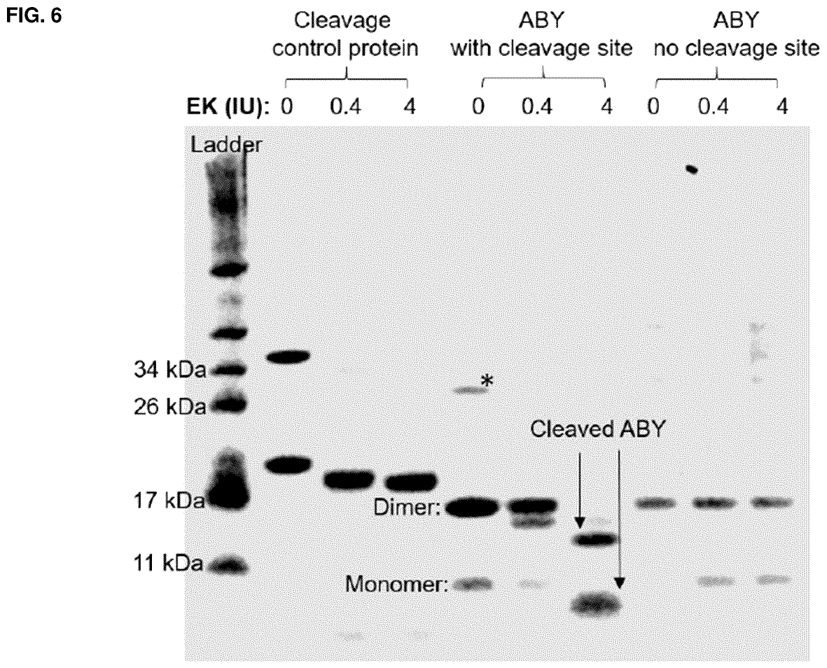

Bacterial tags, such as His-Tag, should be removed from purified proteins being considered for clinical applications as their administration in humans can trigger immune-related adverse events. The ABYACI2 was tested for His-Tag removal in a proof-of-concept enterokinase enzyme-mediated cleavage assay to produce a clinically applicable protein ligand. Incubation of ABY in the cleavage buffer overnight caused dimer formation with a small amount of non-specific aggregation as observed in the SDS-PAGE analysis (FIG. 6); however, the enterokinase enzyme was able to remove the His-Tag from both the dimer and monomer forms of the modified ABY consisting of enterokinase recognition sequence (note the relative reduction in ABY molecular weight after cleavage), but it did not remove the His-Tag from the ABY protein that does not have the recognition sequence. It is possible to minimize the ABY dimer formation by thiol reduction at its cysteine residue prior to incubation with enterokinase, and by decreasing the reaction incubation period (FIG. 1B, lane #3). Based on all these results, the ABYACI 2 was selected as a clinically translatable ligand for further downstream formulations and applications.

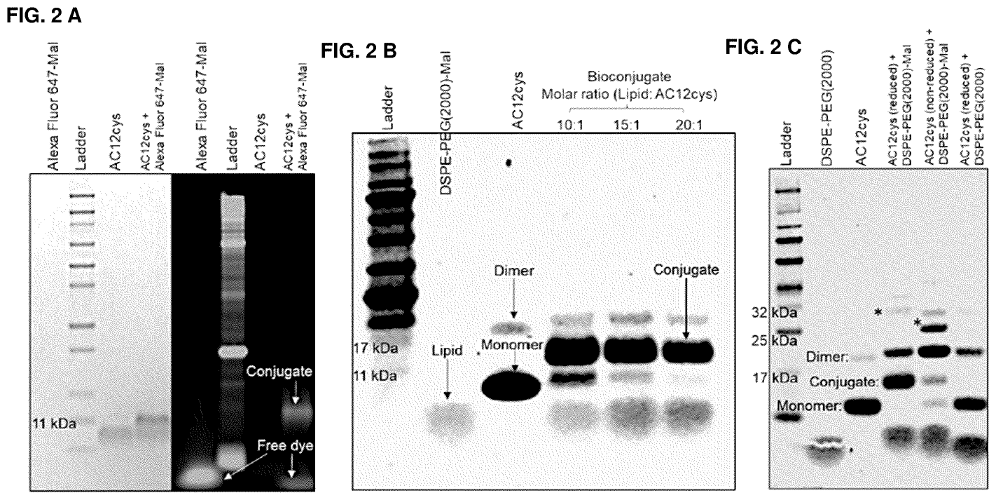

Bioconjugation Optimization and Stability of the ABY-Phospholipid Conjugates. The site-specific conjugation of ABYs with Mal-phospholipids via thiol-maleimide addition reaction yielded >90% conjugation efficiency. Modification with cys with more than one cys moiety in a molecule provides multiple conjugation reaction leads to several derivatives [39]. Regarding the ABY binders (ABYAC2, and ABYACI2), there is no cys residue in both except the one introduced by us at the C-terminus to control the conjugation reactions with only one conjugate derivative. After this modification, the conjugation condition was optimized by using His-Tagged ABYAC12 to test its ability to conjugate with the Mal functional group at neutral pH (7.0). First, the c-terminal cys in the ABY was reduced to activate the thiol group for its conjugation with Alexa Fluor 647-Mal dye. A significant fraction of reduced ABYACI 2 was able to conjugate with the Mal-bearing dye at 10-fold molar excess of the dye as evidenced by fluorescence imaging of the band after resolving in SDS-PAGE gel, whereas no fluorescence band was visualized with the ABY sample devoid of the conjugating dye (FIG. 2A, lane #3). ABY binder with DSPE-PEG-Mal at 1:10 molar ratio yielded a high conjugation efficiency after 2 h of reaction at room temperature (FIG. 2B, lane #4). A homogeneously stained diffused band in FIG. 2B represents the phospholipid of expected molecular weight of 2.9 kDa.

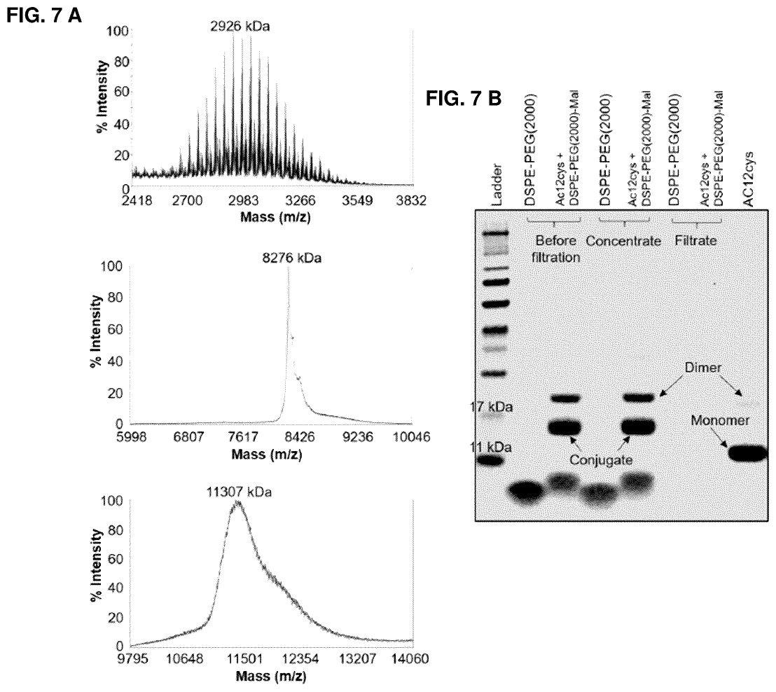

SDS-PAGE analysis in FIG. 2B displays bands corresponding to the conjugates sampled from various molar ratio (1:10, 1:15, 1:20) reactions and compared them with lipid-Mal without any conjugated (FIG. 2B, lane #2) and unconjugated ABY (FIG. 2B, lane #3). While lipid-Mal displays a single band, the reduced ABY shows >98% monomer form with a small fraction of ABY-cys forming dimers during the incubation period. Regarding ABY-reduction, at 1:20 molar ratio, no ABY was leftover as evidenced by no band for monomer, whereas at 1:10, and 1:15 molar ratios, ABY monomer appearance showed proportionally decreased bands corresponding to 17 kDa (FIG. 2B), and the lipid-Mal monomer showed increased band appearance corresponding to 11 kDa (FIG. 2B). These results demonstrate that 20-fold molar excess of lipid-Mal yields 100% conjugation efficiency to form ABY-DSPE-PEG. In addition, MALDI-TOF analysis of ABY-conjugate accounted for an approximate peak size at 11.307 kDa, which is closer to the theoretical summation of molecular weights of DSPE-PEG-Mal lipid monomer (˜2.9 kDa) and ABY (˜8.3 kDa) (FIG. 7A). At pH7, the efficiency of the thiol-maleimide reaction was higher; however, further increases in pH could reduce efficiency by yielding random amine-maleimide reactions [40]. This competing reaction may lead to the formation of undesirable DSPE-PEG-ABY conjugate, which in turn can decrease the chemoselectivity of Mal to the terminal cys on ABY. To confirm the cys site-specificity of the thiol-Mal adducts at neutral pH, the conjugation of DSPE-PEG-Mal was repeated with thiol reduced or non-reduced ABY in an overnight (˜16 h) reaction at 4° C. As expected, a strong band of bioconjugation was observed in sample containing the DSPE-PEG-Mal and reduced ABY monomer, but the longer reaction time also increased the amount of ABY dimers and high molecular weight conjugates (˜30 kDa) (FIG. 2C). Although a minor bioconjugate band (˜11 kDa) was also observed between DSPE-PEG-Mal and non-reduced ABY monomer, a major conjugate band (˜25 kDa size) appeared above the ABY dimer band, which suggests the predominance of amine-Mal adduct formation when reactive thiols from cysteine are not available. The relative increase in the amount of ABY dimer under non-reducing conditions may have contributed to increased Mal reaction with lysines from ABY dimers. Furthermore, the higher molecular weight conjugates (˜30 kDa) also appeared with non-reduced ABY, suggesting that the conjugations under prolonged reaction times lead to Mal-amine reaction. The reduced ABY protein did not form bioconjugates in the reaction samples consisting of control DSPE-PEG without Mal functional groups.

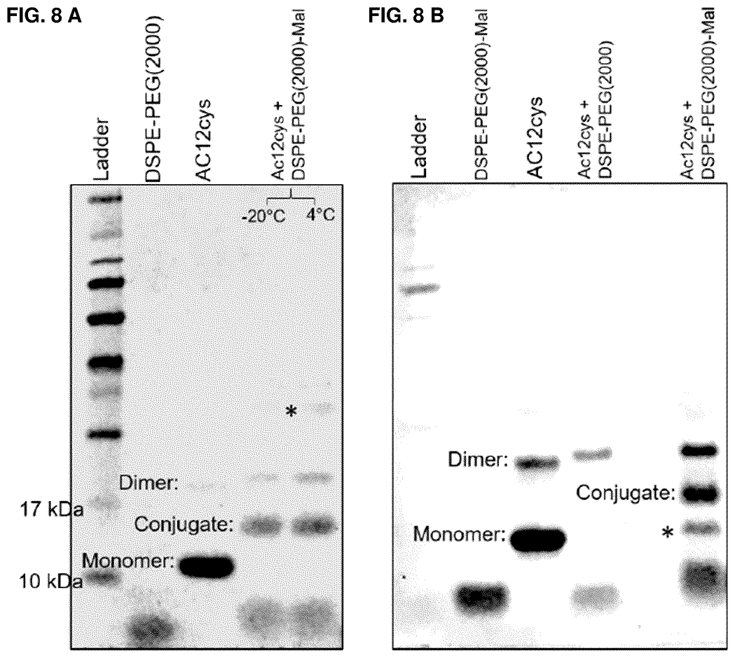

The stability of the DSPE-PEG-ABY was further tested under several storage conditions. Conjugate samples were concentrated and dissolved in water for analysis (FIG. 7B). These samples were stored in two different conditions, at 4° C. or as a lyophilized powder at −20° C. Bulk storage of lyophilized conjugate powder allows DSPE-PEG nanomicelles to be preserved without loss in integrity [41]. Overall, stability testing of DSPE-PEG-ABY indicated that the lyophilized powder conjugate showed no sign of degradation as observed in the SDS-PAGE (FIG. 8A). In addition, the ABYAC12 maintains its target binding activity and does not degrade after multiple freeze-thaw cycles. Similarly, storage of the bioconjugate in water at 4° C. for 2 weeks did not cause any significant dissociation of ABY from DSPE-PEG-ABY due to suspected retro-Michael reaction [42] or spontaneous protein degradation; however, increased non-specific aggregation was observed in this stored micellar solution (FIG. 8A).

Bioconjugation stability is also critical for in vivo applications where phospholipid-Mal exchange from conjugated proteins may occur due to the availability of reactive thiols or abundant amine pools in human plasma (albumin and low molecular weight reactive species) under variable physiological conditions [43]. Although such exchange reactions are shown to negatively affect the long-term therapeutic efficacy of circulating antibody-drug conjugates [43], these reactions may not be rapid enough to affect the diagnostic molecular imaging applications that rely on MBs as contrast agents with short elimination half-life of a few minutes from human circulation [2]. To further assess the in vitro stability of DSPE-PEG-ABY, this conjugate was suspended in a buffer with SDS and reactive amines and stored for 3 weeks at 4° C. Only a small fraction of ABY was observed dissociated from the DSPE-PEG-ABY under this condition, as measured by SDS-PAGE (FIG. 8B), suggesting that the destabilization of bioconjugate depends on storage methods and duration. The optimized thiol-maleimide reaction conditions yielded a stable ligand-phospholipid bioconjugates that can be immediately used or preserved for long-term storage by freeze-dry for their judicious use in MB production.

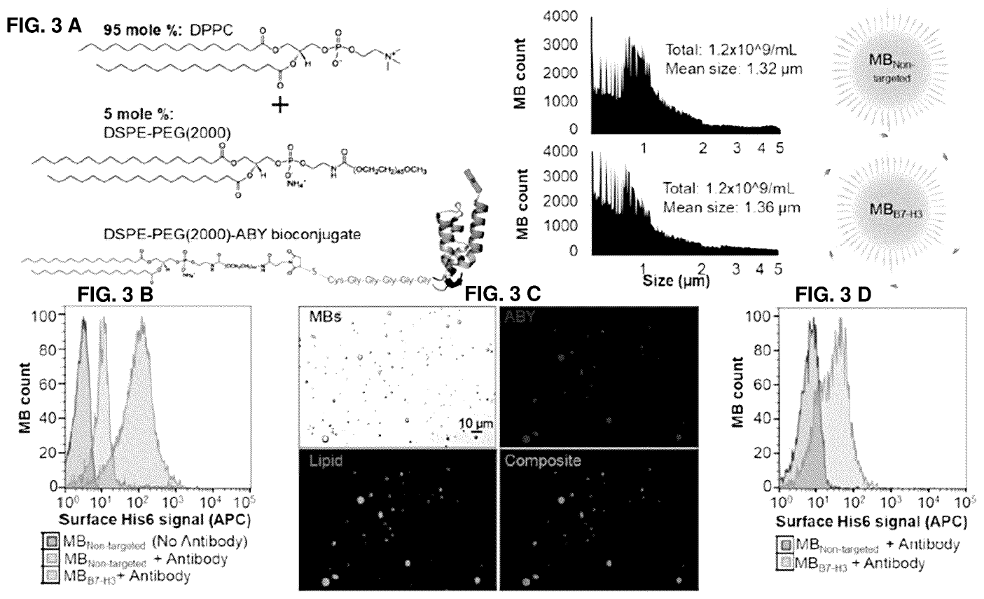

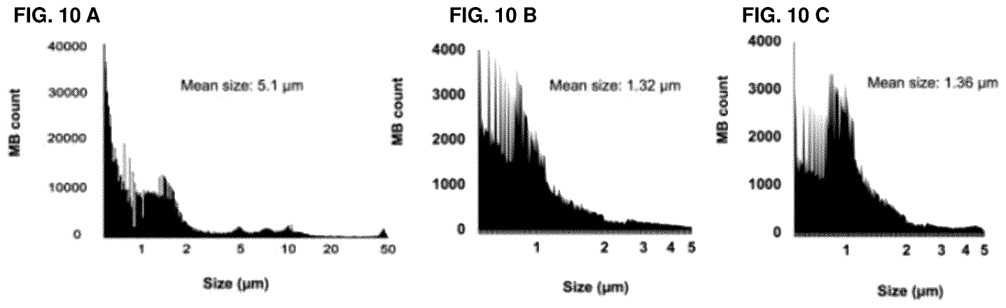

Preparation of Targeted MBB7-H3 with DSPE-PEG-ABY. B7-H3-targeted MBs (MBB7-H3) were prepared by two different methods using the same formulation which include vialmix based amalgamation method, and microfluidic device-based bubble generation, and measured the particles size distribution for comparison (FIG. 10). The microfluidic device capable of producing gas microspheres with precise control over liquid [DSPE-PEG and DPPC liposomal mixture at 1:99 or 5:95 mole % ratio of the lipid components] and gas (perfluorobutane) flow rates [5,44] (FIG. 3A). A 5-9 molar % limit of DSPE-PEG coating is desirable for stable MB generation [45,46], which acts both as an emulsifier and a targeting component. MBs instability may occur due to excessive phase separation of lipids (demixing of DPPC and DSPE-PEG) on MB shell) and result in heterogeneous ligand distribution at higher percentages of DSPE-PEG [47]. DSPE-PEG-biotin emulsifier resulted in a homogeneous distribution of co-localizing ligands on MB surface when DPPC was used as the main phospholipid [23]. As the nature of ligand distribution on phospholipid shell can also affect the functionality of targeted MB and acoustic behavior, the final emulsifying lipid ratio was limited to 5 mole % during MB production. The indicated 1-5 mole % of emulsifier lipid (DSPE-PEG-Mal) micelles used in the targeted MB formulations include the total of DSPE-PEG-Mal bound to ABYACI2 as well as its excess in the bioconjugation reaction mixture (FIG. 2B). The unconjugated lipid consisting of Mal moieties is not active due to the Mal group's susceptibility to rapid hydrolysis and inactivation to corresponding succinamic acid in aqueous solutions [48,49]. Due to the flow-focusing microfluidic chip configuration and a pressure drop at the chip nozzle [5], it was anticipated that flow of the phospholipid suspension containing a mixture of bioconjugate micelles with DPPC liposomes will spontaneously graft associated ligands into the MB shell during the pinch-off stage of MB production by microfluidic system. Particle size quantification showed no significant differences in concentration (˜1.2×10⁹ MBs/mL) and size distribution (mean diameter=1.3 μm; range 0.5-5 μm) between MBB7-H3 and control MBNT (FIG. 3A).

No undesirable particle aggregation was observed due to the inclusion of ABY targeting binder when the MB_{B7-H3} were examined under microscope. For the targeted MB_{B7-H3}, the average number of antibody-based ligands attached per MB is reported to be between 1.0×10⁵ [50] and 2.4×10⁵ [51], whereas small-sized protein ligands can be attached at greater numbers for enhanced binding avidity [52]. Assuming that all reduced ABY molecules are available in a monomer form during the phospholipid bioconjugation steps at an absolute reaction efficiency, a uniformly labeled monodispersed population (based on indicated particle mean size and concentrations) of MB_{B7-H3} should display approximately to a total of 4.0×10⁹ ABY molecules/MB with the use of 5 mole % ratio of bioconjugate mixture. To confirm ligand display on the targeted MBs, flow cytometry analysis was performed using ABY ligands with His-Tag (FIG. 3B). The APC dye conjugated anti-His-Tag antibody was used as an ABY detection antibody. The flow cytometry signal was significantly higher in the MB_{B7-H3} compared to the control MB_{NT} (no binder attached) when 5-mole % of emulsifying lipids were used, suggesting that the bioconjugate integration was successful during microfluidic production. This was further supported by fluorescence microscopy of lipophilic dye-prelabeled MBs showing ABY-associated signal on the surface of the MB_{B7-H3}, but not the MB_{NT} (FIG. 3C). A 1-mole % bioconjugate in hte experiments was not sufficient in detecting ligand display on the targeted MB shell as measured by flow cytometry. The control MBs did not show anti-His-Tag signal enhancement when free unconjugated ABY was supplied in the liquid phase consisting of DSPE-PEG control lipid and DPPC, suggesting that the ABY proteins are covalently tethered to the surface of the targeted MBs through PEG-Mal spacers in bioconjugates, and unconjugated ABY dimers. In contrast, when it is present in low amounts, it does not self-integrate into MB shell during production. To test the robustness of the lipid formulation technique, the production of targeted MB_{B7-H3} was validated by the traditional mechanical agitation method (Vialmix, Lantheus, N. Billerica, MA, USA) using the exact lipid formulation applied to the microfluidic device. As expected, the His-Tag signal corresponding to surface-displayed ABY was also enhanced in these targeted MB_{B7-H3} compared to the control MB_{NT} in the flow cytometry assay (FIG. 3D). These results indicate that molecularly targeted MB_{B7-H3} can be produced in a consistent manner by microfluidic devices using phospholipid and bioconjugate formulations.

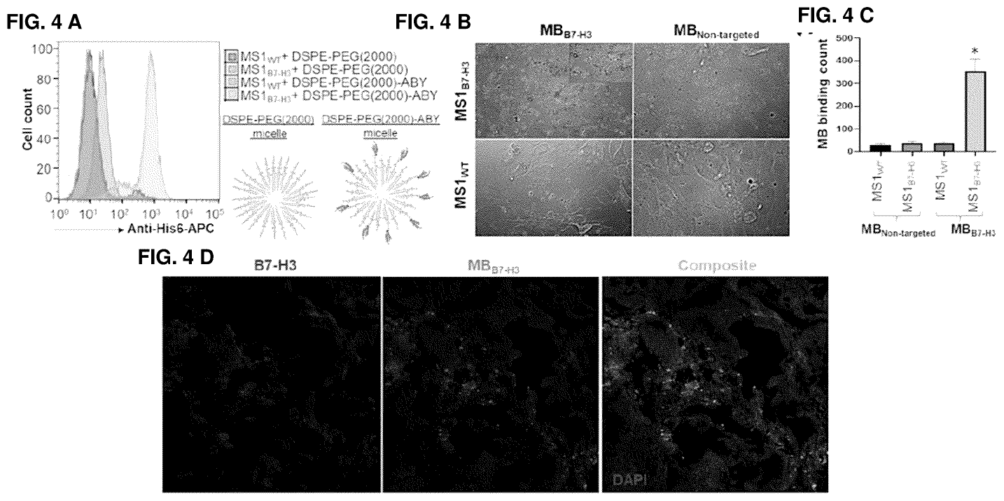

Binding Validation of MBB7-H3 Targeted to Endothelial Cells In Vitro. The retention of target-binding properties of ABY ligand conjugated directly to phospholipid micelles or in the MBB7-H3 shell in vitro in cell culture settings was tested. The target binding of DSPE-PEG-ABY bioconjugate and DSPE-PEG control micelles were tested by flow cytometry in MS1WT and MS1B7-H3 endothelial cells. The bioconjugate binding signal (His-Tag) to MS1B7-H3 cells was higher than its background binding to the wild-type control cells (MS1WT), whereas the binding of the control micelles to both MS1 cell types (MS1B7-H3 and MS1WT) was comparable to the background binding of the bioconjugate micelles (FIG. 4A). These results indicate that the conjugation of ABYAC12 to lipid micelles does not alter its high binding specificity to cellular B7-H3.

The cell-binding ability of the MBB7-H3 was assessed using a monolayer of MS1 cells and quantified the cell-bound MBs by microscopy after the removal of unbound MBs. The average number of MBB7-H3 binding to MS1B7-H3 cells per field of view was significantly higher (354.4±52.3; p<0.05) compared to their binding to the control MS1WT cells (36.2±7.5) (FIG. 4B,C). In contrast, the control MBNT binding to both cell types were comparable and significantly (p<0.05) lower than the binding of MBB7-H3 to MS1B7-H3 cells (37.7±7.8 for MS1B7-H3 and 28.3±6.7 for MS1WT cells), indicating a target-specific binding performance of MBB7-H3 in vitro.

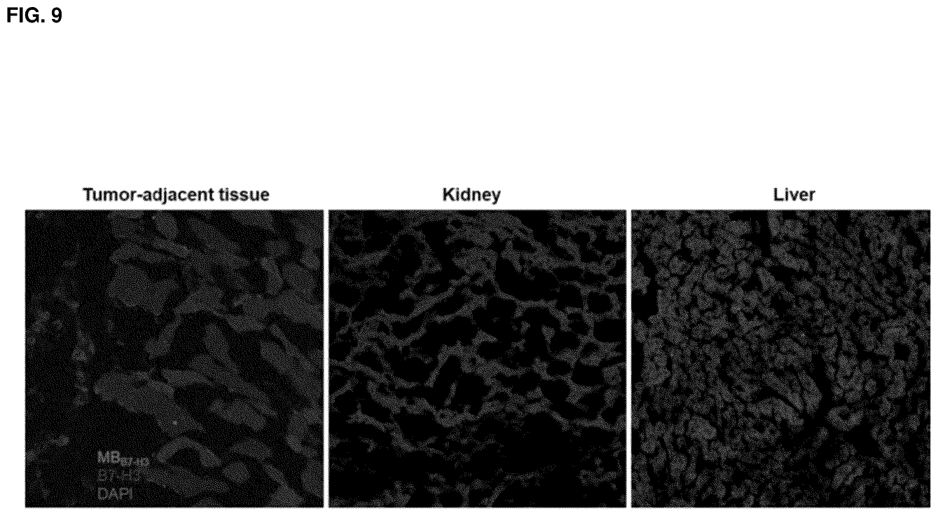

Binding Validation of MBB7-H3 Targeted to Tumor Vascular Endothelial Cells In Vivo. Intravascular target-binding validation of fluorescently labeled MBs (green) was performed in a transgenic (FVB/N-Tg(MMTV-PyMT)634 Mul/J) mouse with mammary tumors by bolus injection of MBB7-H3 via tail vein. Ex vivo analysis of tumor tissues after MBB7-H3 administration showed higher level of vascular B7-H3 (red; FIG. 4D) in the mammary tumors as measured by immunofluorescence staining [53]. B7-H3 signal was not detected in surrounding non-tumor tissues (FIG. 9). MBB7-H3 signal overlapped with anti-B7-H3 antibody-based immunostaining results as imaged by confocal microscopy, indicating that the MBB7-H3 is localized within the B7-H3-positive tumor vasculature (FIG. 4D). Under the influence of hemodynamic forces in vivo, MBB7-H3 target binding activity is expected to be heterogeneous as observed in microscopic images of tumor tissue areas with B7-H3-positive vessels but no MB signal toward the core of larger tumors with few blood vessels. This result suggests that target binding kinetics for MBs could be more pronounced toward the tumor-host interface with abundant blood vessels and associated contrast agent signal for molecular imaging. This, in turn, may depend on the tumor perfusion heterogeneity as well as other characteristics such as tumor size and type [54]. In contrast, MBB7-H3 signal was not present in other highly perfused normal organs such as kidneys, which lack B7-H3 expression in their blood vessels (FIG. 9). While the vascular B7-H3 signal was also absent in the liver, a diffused MB signal in microscopy imaging was observed. Liver is a highly vascularized organ and is known to rapidly remove nanobubbles/MBs from the circulation through sinusoids and the tissue-resident phagocytes [55]. This is consistent with prior observations showing the distribution properties of MBs and their clearance primarily by the liver [56]. Together, the results indicate that the targeted MBs produced by microfluidics can bind specifically to their endothelial target-receptor in vitro and tumor-associated vasculature in vivo.

The use of engineered protein scaffolds with terminally expressed cysteine (affibodies, single-chain variable fragments, diabodies, etc.) to form phospholipid bioconjugates with variations of thiol conjugation chemistry [59,60] may further improve process control and batch reproducibility compared to post-labeling techniques for targeted MB production.

In summary, a novel ABY binder protein has been identified and it was engineered with cystine at C-terminus followed by a 5-glycine linker (GGGGGC) for the site-specific bioconjugation to lipid moiety for MB preparation. In addition, an enterokinase enzyme cleavable linker was added prior to His-Tag so that it can be removed after ABY protein purification. This ABY was evaluated for its binding affinity and optimized the site-specific conjugations efficiency with DSPE-PEG to form targeting ABY-DSPE-PEG. This ABY-lipid was used to link B7-H3 for targeted MBs (MB_{B7-H3}). This product was further evaluated for its binding affinity in both in vitro and in vivo studies. Finally, a reproducible standardized method was established for generating TMBs using a microfluidic system for on-demand preparation of MBs with the potential for human studies. A rigorous quality check was performed of the engineered ABY binder, bioconjugation chemistry, and lipid formulations, while the ligand, bioconjugate, and targeted MBs were evaluated for target binding using various in vitro and in vivo studies. The in vivo efficacy and stability of these contrast agents will be examined by ultrasound molecular imaging applications in the future studies. In summary, MB_{B7-H3} is used as an ultrasound contrast agent for the diagnosis of human solid tumors expressing vascular B7-H3.

The methods of the present disclosure provide specific advantages over existing methods. Existing methods for targeting microbubbles apply bioconjugation after microbubble production, which negatively affects labeling uniformity and batch-to-batch reproducibility. Methods that apply bioconjugates prior to microbubble production are formed with amine conjugation strategy. This strategy has the disadvantage of not having robust site specificity. The cysteine-maleimide conjugation approach allows site-specific conjugation of proteins to phospholipids, and it is often used to form antibody drug conjugates approved by the FDA. Additionally, site-specific bioconjugation with a terminal cysteine reduces the possibilities for altered protein activity that may, otherwise, arise due to random conjugations at multiple available amines in proteins. The terminal cysteine can be easily added to small protein ligands (example: affibody, single-chain variable fragments etc.) by molecular engineering or gene synthesis methods. Preparing phospholipid micellar bioconjugates by this alternative approach contributes to the consistency of re-creating targeted microbubbles against novel disease biomarkers. Additionally, the use of small engineered proteins, as opposed to bigger molecules such as antibodies, with phospholipids is economical and scalable for clinical applications. Lastly, the use of phospholipid-protein bioconjugates in producing microbubbles via microfluidic chips is novel and can have significant positive impact in achieving control over production consistency of clinically translatable ultrasound contrast agents.

### Materials and Methods

Reagents and Chemicals. All lipids used for microbubble (MB) formulation were purchased in powdered forms from Avanti Polar Lipids and stored at −20° C. (Avanti Polar Lipids Inc., Alabaster, AL, USA). The lipid 1,2-distearoyl-sn-glycero-3-phosphoethanolamine-N-[methoxy(polyethylene glycol-2 kDa)] (DSPE-PEG) without or with maleimide group (DSPE-PEG-Mal), along with 1,2-dipalmitoyl-sn-glycero-3-phosphocholine (DPPC) were used for the synthesis process. The following other chemicals of analytical grades for MB production were also used: Glycerol (Sigma Aldrich, Burlington, MA, USA), Sodium Chloride (NaCl; Fisher Bioreagents), Perfluorobutane (FluoroMed Inc., Round Rock, TX, USA), Perfluorohexane (Sigma Aldrich, Burlington, MA, USA), and UltraPure Distilled Water (Invitrogen, Waltham, MA, USA).

Cell Culture. MILE SVEN 1 mouse vascular endothelial cells of wild-type (MS1_{WT}) were obtained from American Type Culture Collection (ATCC; CRL2279) and stably transfected with human B7-H3 expression vector to generate MS1_{B7-H3} cells as described previously [61]. DMEM (Corning Inc., Glendale, AZ, USA) cell culture media containing 5% fetal bovine serum and 100 units/mL of penicillin and 100 μg/mL of streptomycin was used to culture MS1 cells. Similarly, 4T1 (ATCC; CRL2539) mouse breast cancer cells and MDA-MB-231 (ATCC; HTB-26) human breast cancer cells were maintained in DMEM supplemented with 10% fetal bovine serum, 100 units/mL of penicillin, and 100 μg/mL of streptomycin.

Protein Modification, Expression, and Purification. ABY proteins bind to the extracellular domain of human B7-H3 receptor were previously identified using a yeast display library [61]. Parental AC2 (K_{d}=310±100 nM) binder was modified by site-directed mutagenesis to identify an AC12 binder with single digit nanomolar affinity (K_{d}=0-9±0.6 nM) [61]. A C-terminal cysteine (cys) residue tethered to a pentaglycine bridge (Gly-Gly-Gly-Gly-Gly-Cys) was introduced for site-specific bioconjugation, and an N-terminal His-Tag sequence for affinity chromatography isolation with a linking enterokinase cleavage site for His-Tag removal (FIG. 1a). The purified PCR amplicons were subcloned into pET-22b plasmid vector at Nhel and BamHI restriction cloning sites for protein expression and purification in E. coli. The sequence confirmed clone using a T7 terminator primer (Sequetech, CA, USA) was used for protein expression.

The two ABY binders (AC2 and AC12) transformed into BL21 E. coli(NEB) cells were used for protein purification. The bacterial colonies were outgrown in lysogeny broth supplemented with Ampicillin antibiotics for overnight, and induced for expression by treating with 200 μM of isopropyl β-D-1-thiogalactopyranoside for four to five hours at 37° C. Bacterial cells were pelleted and lysed in lysis buffer containing 3.4 mM of NaH₂PO₄, 46 mM of Na₂HPO₄, 25 mM of imidazole, protease inhibitors (all reagents were purchased from Fisher Bioreagents, Hampton, NH, USA), 0.5 M of NaCl, 0.7 M of glycerol, and 5 of mM CHAPS (Sigma Aldrich, Burlington, MA, USA). ABY was purified by Ni-NTA affinity chromatography column (HisTrap-1 mL; GE Healthcare). Purified ABYs were desalted using 7 kDa molecular weight cut-off Zeba spin columns (Thermo Scientific, Waltham, MA, USA). ABYs were lyophilized overnight using a vacuum freeze dryer (Labconco) and stored at −20° C. until further use. Ligands were validated for purity and size by 4-12% SDS-PAGE (Novex™, Thermo Scientific, Waltham, MA, USA) by loading 5 μg of protein in Laemelli buffer (Bio-Rad Laboratories, Hercules, CA, USA). The gels were stained with Coomassie G-250 dye (SimplyBlue SafeStain, Invitrogen, Waltham, MA, USA) for 1 h, followed by overnight destaining in water for visualization of protein for purity. Protein bands were visualized by gel imaging at 700 nm wavelength in an Odyssey Imaging System (LI-COR Bioscience, Lincoln, NE, USA). ABY was further characterized by matrix assisted laser desorption ionization-time of flight mass spectrometry (MALDI-TOF) to confirm sample purity and mass.

Biotinylation of ABY. ABY proteins were biotinylated with NHS-PEG₄-Biotin (ThermoFisher Scientific, Waltham, MA, USA) using 1:1.6 molar ratio [61] of ABY: NHS-PEG₄-Biotin. Briefly, NHS-PEG₄-Biotin was dissolved in PBS (pH 7.2) and immediately mixed with 25 μL of ABY (1 mg/mL in UltraPure Water) to achieve 1.6-fold molar excess of biotin to the ABY protein and incubated at room temperature for 30 min. Unconjugated excess biotin was cleaned by passing through a 7 kDa molecular weight cut-off Zeba spin column.

His-Tag Removal by Enzymatic Cleavage. The N-terminal poly histidine tag (6×Histidine; His-Tag) was cleaved off from the ABY protein by recombinant bovine enterokinase enzyme (GenScript, Piscataway, NJ, USA) at the cleavage site (-Asp-Asp-Asp-Asp-Lys sequence) designed at the upstream of the ABY sequence. Briefly, freeze-dried ABY was resuspended in UltraPure Water at 1 μg/μL concentration. A total of 10 μg of ABY was added to the cleavage buffer containing enterokinase enzyme (0.4 IU or 4 IU) and incubated at room temperature for 16 h [62]. A total of 40 kDa of control protein supplied with the kit was used as a positive control while ABY expressed from the original plasmid without the N-terminal His-Tag, and cleavage site was used as negative control for the cleavage assay.

Phospholipid-Ligand Bioconjugation (FIG. 5). ABY was conjugated to Alexa Fluor 647-maleimide (Mal) dye (ThermoFisher Scientific, Waltham, MA, USA) in UltraPure Water at neutral pH. Prior to Mal dye conjugation, ABY was treated with TCEP.HCl (Thermo Scientific, Waltham, MA, USA) at 1:10 mole ratio for 30 min in UltraPure Water at room temperature to reduce any possible ABY dimer with Cys-Cys bond. Ten-fold molar excess of Alexa Fluor 647-Mal was added to the reduced ABY binder and incubated for 2 h at room temperature. The ABY-Alexa Fluor 647 conjugation was confirmed by fluorescence imaging of SDS-PAGE gels in the Odyssey Imaging System (LI-COR Biosciences, Lincoln, NE, USA).

DSPE-PEG-Mal was conjugated to reduced ABY in UltraPure Water at neutral pH to generate the DSPE-PEG-ABY. Briefly, ABY was first reduced as described earlier and mixed with DSPE-PEG-Mal micelles that were prepared in UltraPure Water above the critical micellar concentration (0.36 mg in 500 μL water). Dispersions of DSPE-PEG in pure water have low aggregation number and spherical core shape [63]. Conjugation reaction was optimized by performing various molar ratios of DSPE-PEG-Mal: ABY (20:1, 15:1, 10:1), respectively, for 2 h at room temperature. The effect of incubation time and temperature were also tested by performing these bioconjugation reactions overnight at 4° C. with a 20-fold molar excess of phospholipids. The resulting conjugate was purified from TCEP and other impurities using an ultracentrifugal filter column (Amicon, 10 kDa cut-off, Burlington, MA, USA). The concentrated bioconjugate in water was stored at 4° C. for the short-term, or at −20° C. as a lyophilized powder prepared by vacuum freeze-drying process for long-term.

Formation of thiol adducts can be detected by gel shift assays [48]. The bioconjugate reaction between ABY and the DSPE-PEG-Mal was evaluated by SDS-PAGE analyses by comparing the samples with individual reaction components (ABY or DSPE-PEG-Mal) against the final product of DSPE-PEG-ABY. Approximately 5 μg of ABY and 20 μg of micellar phospholipid equivalents were loaded in Laemelli buffer for SDS-PAGE analysis (NuPAGE Bis-Tris gels with neutral pH) to compare the bioconjugates. Protein and micellar phospholipid bands were visualized by Coomassie G-250 dye staining and imaging as described above.

For the bioconjugate stability tests, DSPE-PEG-ABY bioconjugate was stored in a Laemelli buffer with SDS and competing primary amines (15 mM Tris.HCl solution at neutral pH) for 3 weeks at 4° C., and ABY dissociation from the bioconjugate was assessed by SDS-PAGE analysis. The bioconjugates stored in water at 4° C. or as lyophilized powder at −20° C. (after resuspension in water) were also examined for degradation by the SDS-PAGE analysis.

Flow Cytometry Analysis. ABY binder was tested for its binding ability to MS1_{WT}, MS1_{B7-H3}, MDA-MB-231, and 4T1 cells. Approximately 1×10⁶ cells of each cell type were incubated with or without biotinylated ABY (0 and 1 μM) for one hour at room temperature, then washed 3 times in PBS containing 0.1% BSA, and incubated with streptavidin-Alexa Fluor 647 dye (Thermo Scientific) for 30 min. A control group of cells incubated only with streptavidin-Alexa Fluor 647 dye (without ABY) was used as a control for non-specific dye binding to cell surface. Cells were also tested with anti-B7-H3 antibody with APC dye (BioLegend) known as a positive control to bind for B7-H3. Next, cells were washed 3 times in 1% PBSA to remove unbound dye. A Guava easyCyte flow cytometer was used to analyze these cells using FlowJo 2.0 software for the histogram comparison of cell bound ABY signal.

In addition to ABY-PEG-dye, ABY-conjugate micelles were also tested for its target binding on the MS1 cells. 25 μL of ABY-DSPE-PEG was incubated with 0.5×10⁶ MS1_{WT} or MS1_{B7-H3} cells in 100 μL of PBS (corresponds to 4 μM ABY) for 1 h at room temperature. Cells incubated with DSPE-PEG micelles alone were used as a negative control. The cells were washed 3 times in 1% PBS to remove unbound micelles and incubated with anti-His-Tag-APC secondary antibody (BioLegend, San Diego, CA, USA) for 30 min. The cells were then washed 3 times in 1% PBS and analyzed using flow cytometry.

Preparation of Targeted Microbubbles by a Microfluidic System. The Horizon Microbubble Maker system (purchased from the University of Leeds, U.K.) was used to generate MBs. The Horizon system is a microfluidics-based system for producing uniform and reproducible distributions of polydisperse and monodisperse microbubbles by mixing lipids and gases via (interchangeable) microfluidic cartridges. This computer-controlled system can produce bubble of different sizes by adjusting the flow rate of lipid mixture, gas pressure, and perfluorobutane level (see FIG. 1). Similarly, the system can also be operated using cartridges of various designs to prepare MBs of different sizes and properties. A detailed design and operation of the Horizon system is reported by Abou-Saleh et al. [5, 6].

A standard operation procedure (SOP) established by the manufacturer was followed to prepare MBs [5]. DPPC was prepared based unilamellar liposomes from a thin film of phospholipids prepared in a glass vial by evaporating organic solvents from the lipid mixture by passing steady flow of N₂ gas over this mixture. This lipid film was redissolved in saline by probe tip sonication (Branson SLPe Digital Sonifier) on ice. After sonication the liposome size was determined by Dynamic Light Scattering (DLS, Malvern Zetasizer, Malvern analytical) and Nanoparticle tracking analysis (Nanosight, NS300, Malvern). The B7-H3-targeted MBs (MB_{B7-H3}) were prepared from the mixture of DSPE-PEG-ABY and DPPC (2 mg/mL of lipids containing DSPE-PEG-ABY and DPPC at 1:99 or 5:95% molar ratio) in final buffer containing 4 mg/mL of NaCl, 1% perfluorohexane and 1% glycerol (Sigma Aldrich, Burlington, MA, USA) in UltraPure Water. Briefly, DPPC thin film was hydrated in 0.5 mL of buffer in a glass vial followed by 30 min heating at 55° Celsius for lipid phase transition, 1 min vortexing, and tip-sonication on ice until the lipid solution turned transparent by visual inspection. A total of 0.5 mL of the DSPE-PEG-ABY bioconjugate solution was added to 0.5 mL of DPPC liposome buffer solution and mixed. A total of 10 μL of perfluorohexane was added to the resulting 1 mL of phospholipid mixture and vortexed immediately before MB production.

The microfluidic chips in the Horizon system were supplied with perfluorobutane gas (FluoroMed, Inc, Round Rock, TX, USA) at 1000 mbar pressure through a gas inlet channel and the 1 mL of lipid mixture was introduced in aqueous phase through two opposing inlet channels at 100 μL/minute flow rate, with the flow focused through a chip nozzle to produce MBs. Control MBs (Non-targeted=MB_{NT}) were prepared with the lipid mixture containing DSPE-PEG without Mal group and DPPC at the indicated mole ratios described above. MB count and particle size were determined by Accusizer 770A (Particle Sizing Systems) and the values were compared to MBNT.

Validation of ABY Display on Targeted Microbubbles. The DSPE-PEG-ABY conjugate amalgamated into the DPPC MBs was confirmed by flow cytometry with an anti-His-Tag-APC antibody. 5 μL of antibody was added to 100 μL of MBs (MB_{B7-H3} or MB_{NT}) containing 1×10⁸ particles and the mixture was incubated at room temperature for 1 h. MBs were washed 3× in 500 μL of PBS using a microcentrifuge at 300 g for 3 min. After each wash, the upper milky layer of floating MBs was carefully separated by removing the liquid wash with a syringe needle and resuspending MBs in fresh PBS. ABY (ABY_{NoHis-Tag}) displayed on the surface of MB_{B7-H3} was detected by flow cytometry (Guava easyCyte) of MB-bound anti-His-Tag-APC antibody and compared to MB_{NT} background signal or MB_{B7-H3} without antibody incubation. MB-ABY fluorescence signal was also analyzed using confocal microscopy (DMi8, Leica) for both targeted and non-targeted MBs (pre-labeled with CellMask Green, ThermoFisher Scientific, Waltham, MA, USA) at 20× magnification. Acquired images were analyzed using ImageJ 1.52a software (NIH, Baltimore, MD, USA).

In Vitro Binding Assay of MB to B7-H3. MS1_{WT} or MS1_{B7-H3} cells were cultured (3×10⁵ cells/well) on glass coverslips (VWR) placed on the bottom of a 6-well plate (Corning Inc, Glendale, AZ) with DMEM media (2 mL/well). After three days, non-adherent cells were removed from the wells by gently washing with warm PBS (2 mL/well; 3×) followed by application of a hydrophobic barrier (Super Pap Pen, Daido Sangyo Co., Ltd, Tokyo, Japan) around the coverslips in each well. A total of 1×10⁸ MBs (MB_{B7-H3} or MB_{NT}) were added immediately to the top of the coverslips in warm PBS (100 μL/coverslip) to allow the MBs to bind to cells. After 30 min incubation at 37° C., cells were gently washed with warm PBS (2 mL/well; 3×) to remove all the unbound MBs in the wells. Cells were fixed with 4% paraformaldehyde solution for 10 min and washed 3× in PBS. The dried coverslips were mounted onto glass slides with Toluene solution (Fisher Scientific, Waltham, MA, USA) and kept at 4° C. for storage. Glass slides with cell-bound MBs were imaged under brightfield microscopy (Leica DM4, 20× magnification). The number of MBs attached to MS1_{WT} or MS1_{B7-H3} cells (9 areas/coverslip) were quantified by image processing in ImageJ 1.52a. Briefly, image files were converted to 8-bit format and cell-bound MBs were identified automatically as bright circular spots by the image processing software (Spot Caliper) plugin [64] implemented via ImageJ. This experiment was repeated three times with different batches of MBs.

MBs Binding to B7-H3 Target Expressed in Mouse Tumor-Associated Blood Vessels after Intravenous Injection by Ex Vivo Immunostaining Analyses. Animal experiments were approved by the Institutional Administrative Panel on Laboratory Animal Care at Stanford University. MB_{B7-H3} binding to its molecular target was validated in a transgenic mouse model (FVB/N-Tg(MMTV-PyMT)634 Mul/J) expressing B7-H3 in its tumor-associated vasculature [53]. A lipophilic staining reagent (CellMask Green, ThermoFisher Scientific, Waltham, MA) was used to label MBs with a fluorescent dye (Fluorescence excitation/emission maxima: 522/535 nm) prior to administration into mouse. Mammary tumors of the mouse were pre-confirmed by gene sequence testing for positive expression in all its glands (3-7 mm in diameter). Tumor positive mice (N=10 tumors per animal) was anesthetized with a constant supply of 2% isoflurane in oxygen. A 200 μL bolus injection of MB_{B7-H3} containing 1×10⁸ bubbles was slowly injected intravenously through the tail vein of the mice (Catheter with 27 g butterfly needle; FUJIFILM VisualSonics, Toronto, ON, Canada) followed by 20 μL of saline flush, and MBs were allowed to attach to the molecular target, B7-H3, for 5 min. The mouse was sacrificed and infused with 5 mL of 4% paraformaldehyde (Santa Cruz Biotechnology, Dallas, TX, USA) via cardiac puncture to wash off unbound microbubbles from the circulation and to simultaneously preserve tumor tissues based on a whole animal perfusion fixation method [65]. Mouse organs and tumor tissues were surgically extracted (mammary tumor tissues, liver, kidneys) and frozen in Optimum Cutting Temperature (OCT) media (Tissue-Tek) for further processing.

The frozen tissues were sectioned (10 μm thickness) on glass slides using a Cryostat (Leica Biosystems, Wetzlar, Germany) and processed for immunofluorescence staining, as described previously [53]. Briefly, tissue sections were rinsed with PBS for 5 min to remove the OCT media. This was followed by washing for 3 times in PBS and blocking in 5% normal goat serum in PBS for one hour at room temperature. The tissue slices were further incubated with a rat anti-mouse B7-H3 primary antibody (Abcam, Waltham, MA, USA) at a dilution of 1:50 overnight at 4° C. followed by incubation with Alexa Fluor 594 goat anti-rat secondary antibody (Invitrogen, Waltham, MA, USA) at 1:300 dilution for 30 min at room temperature. The fluorescent images, representing MB localization (green channel) and B7-H3 expression (red channel), were acquired by confocal microscopy with 20× magnification (LSM 510 Meta confocal microscope, Carl Zeiss, Jena, Germany) and composite images were created in ImageJ [66].

Statistical Analysis. The number of MBs in the in vitro cell binding assay groups is presented as average±standard error of mean. Groups for MB binding are compared against each other by unpaired t-test with a p-value<0.05 considered as statistically significant.

### REFERENCES

1. Klibanov, A. L. Ultrasound Contrast: Gas Microbubbles in the Vasculature. Investig. Radiol. 2021, 56, 50-61.
2. Willmann, J. K.; Bonomo, L.; Testa, A. C.; Rinaldi, P.; Rindi, G.; Valluru, K. S.; Petrone, G.; Martini, M.; Lutz, A. M.; Gambhir, S. S. Ultrasound Molecular Imaging With BR55 in Patients With Breast and Ovarian Lesions: First-in-Human Results. J. Clin. Oncol. 2017, 35, 2133-2140.
3. Smeenge, M.; Tranquart, F.; Mannaerts, C. K.; de Reijke, T. M.; van de Vijver, M. J.; Laguna, M. P.; Pochon, S.; de la Rosette, J.; Wijkstra, H. First-in-Human Ultrasound Molecular Imaging With a VEGFR2-Specific Ultrasound Molecular Contrast Agent (BR55) in Prostate Cancer: A Safety and Feasibility Pilot Study. Investig. Radiol. 2017, 52, 419-427.
4. Talu, E.; Hettiarachchi, K.; Zhao, S.; Powell, R. L.; Lee, A. P.; Longo, M. L.; Dayton, P. A. Tailoring the size distribution of ultrasound contrast agents: Possible method for improving sensitivity in molecular imaging. Mol. Imaging 2007, 6, 384-392.
5. Abou-Saleh, R. H.; Armistead, F. J.; Batchelor, D. V. B.; Johnson, B. R. G.; Peyman, S. A.; Evans, S. D. Horizon: Microfluidic platform for the production of therapeutic microbubbles and nanobubbles. Rev. Sci. Instrum. 2021, 92, 74105.
6. Carugo, D.; Browning, R. J.; Iranmanesh, I.; Messaoudi, W.; Rademeyer, P.; Stride, E. Scaleable production of microbubbles using an ultrasound-modulated microfluidic device. J. Acoust. Soc. Am. 2021, 150, 1577.
7. Bachawal, S.; Bean, G. R.; Krings, G.; Wilson, K. E. Evaluation of ductal carcinoma in situ grade via triple-modal molecular imaging of B7-H3 expression. NPJ Breast Cancer 2020, 6, 14.
8. Mulvana, H.; Browning, R. J.; Luan, Y.; de Jong, N.; Tang, M. X.; Eckersley, R. J.; Stride, E. Characterization of Contrast Agent Microbubbles for Ultrasound Imaging and Therapy Research. IEEE Trans. Ultrason. Ferroelectr. Freq. Control. 2017, 64, 232-251.
9. Anderson, C. R.; Rychak, J. J.; Backer, M.; Backer, J.; Ley, K.; Klibanov, A. L. scVEGF microbubble ultrasound contrast agents: A novel probe for ultrasound molecular imaging of tumor angiogenesis. Investig. Radiol. 2010, 45, 579-585.
10. Yeh, J. S. M.; Sennoga, C. A.; McConnell, E.; Eckersley, R.; Tang, M. X.; Nourshargh, S.; Seddon, J. M.; Haskard, D. O.; Nihoyannopoulos, P. A Targeting Microbubble for Ultrasound Molecular Imaging. PLoS ONE 2015, 10, e0129681.
11. Bam, R.; Daryaei, I.; Abou-Elkacem, L.; Vilches-Moure, J. G.; Meuillet, E. J.; Lutz, A.; Marinelli, E. R.; Unger, E. C.; Gambhir, S. S.; Paulmurugan, R. Toward the Clinical Development and Validation of a Thy1-Targeted Ultrasound Contrast Agent for the Early Detection of Pancreatic Ductal Adenocarcinoma. Investig. Radiol. 2020, 55, 711-721.
12. Punjabi, M.; Xu, L.; Ochoa-Espinosa, A.; Kosareva, A.; Wolff, T.; Murtaja, A.; Broisat, A.; Devoogdt, N.; Kaufmann, B. A. Ultrasound Molecular Imaging of Atherosclerosis With Nanobodies: Translatable Microbubble Targeting Murine and Human VCAM (Vascular Cell Adhesion Molecule) 1. Arterioscler. Thromb. Vasc. Biol. 2019, 39, 2520-2530.
13. Liu, X.; Sun, J.; Gao, W. Site-selective protein modification with polymers for advanced biomedical applications. Biomaterials 2018, 178, 413-434.
14. Park, J. W.; Hong, K.; Kirpotin, D. B.; Colbern, G.; Shalaby, R.; Baselga, J.; Shao, Y.; Nielsen, U. B.; Marks, J. D.; Moore, D.; et al. Anti-HER2 immunoliposomes: Enhanced efficacy attributable to targeted delivery. Clin. Cancer Res. 2002, 8, 1172-1181.
15. Pillai, R.; Marinelli, E. R.; Fan, H.; Nanjappan, P.; Song, B.; von Wronski, M. A.; Cherkaoui, S.; Tardy, I.; Pochon, S.; Schneider, M.; et al. A phospholipid-PEG2000 conjugate of a vascular endothelial growth factor receptor 2 (VEGFR2)-targeting heterodimer peptide for contrast-enhanced ultrasound imaging of angiogenesis. Bioconjugate Chem. 2010, 21, 556-562.
16. Zhang, B.; Vidanapathirana, S. M.; Greineder, C. F. Site-Specific Modification of Single-Chain Affinity Ligands for Fluorescence Labeling, Radiolabeling, and Bioconjugation. In Peptide Conjugation; Methods in Molecular Biology; Humana: New York, NY, USA, 2021; Volume 2355, pp. 163-173.
17. Snipstad, S.; Vikedal, K.; Maardalen, M.; Kurbatskaya, A.; Sulheim, E.; Davies, C. d. L. Ultrasound and microbubbles to beat barriers in tumors: Improving delivery of nanomedicine. Adv. Drug Deliv. Rev. 2021, 177, 113847.
18. Natarajan, A.; Xiong, C. Y.; Albrecht, H.; DeNardo, G. L.; DeNardo, S. J. Characterization of site-specific ScFv PEGylation for tumor-targeting pharmaceuticals. Bioconjugate Chem. 2005, 16, 113-121.
19. Peyman, S. A.; Abou-Saleh, R. H.; McLaughlan, J. R.; Ingram, N.; Johnson, B. R. G.; Critchley, K.; Freear, S.; Evans, J. A.; Markham, A. F.; Coletta, P. L.; et al. Expanding 3D geometry for enhanced on-chip microbubble production and single step formation of liposome modified microbubbles. Lab Chip 2012, 12, 4544-4552.
20. Yang, S.; Wei, W.; Zhao, Q. B7-H3, a checkpoint molecule, as a target for cancer immunotherapy. Int. J. Biol. Sci. 2020, 16, 1767-1773.
21. Segers, T.; Kruizinga, P.; Kok, M. P.; Lajoinie, G.; de Jong, N.; Versluis, M. Monodisperse Versus Polydisperse Ultrasound Contrast Agents: Non-Linear Response, Sensitivity, and Deep Tissue Imaging Potential. Ultrasound Med. Biol. 2018, 44, 1482-1492.
22. Langeveld, S. A. G.; Meijlink, B.; Beekers, I.; Olthof, M.; van der Steen, A. F. W.; de Jong, N.; Kooiman, K. Theranostic Microbubbles with Homogeneous Ligand Distribution for Higher Binding Efficacy. Pharmaceutics 2022, 14, 311.
23. Langeveld, S. A. G.; Schwieger, C.; Beekers, I.; Blaffert, J.; van Rooij, T.; Blume, A.; Kooiman, K. Ligand Distribution and Lipid Phase Behavior in Phospholipid-Coated Microbubbles and Monolayers. Langmuir 2020, 36, 3221-3233.
24. Chapoval, A. I.; Ni, J.; Lau, J. S.; Wilcox, R. A.; Flies, D. B.; Liu, D.; Dong, H.; Sica, G. L.; Zhu, G.; Tamada, K.; et al. B7-H3: A costimulatory molecule for T cell activation and IFN-gamma production. Nat. Immunol. 2001, 2, 269-274.
25. Hofmeyer, K. A.; Ray, A.; Zang, X. The contrasting role of B7-H3. Proc. Natl. Acad. Sci. USA 2008, 105, 10277-10278.
26. Xu, H.; Cheung, I. Y.; Guo, H. F.; Cheung, N. K. MicroRNA miR-29 modulates expression of immunoinhibitory molecule B7-H3: Potential implications for immune based therapy of human solid tumors. Cancer Res. 2009, 69, 6275-6281.
27. Lee, Y. H.; Martin-Orozco, N.; Zheng, P.; Li, J.; Zhang, P.; Tan, H.; Park, H. J.; Jeong, M.; Chang, S. H.; Kim, B. S.; et al. Inhibition of the B7-H3 immune checkpoint limits tumor growth by enhancing cytotoxic lymphocyte function. Cell Res. 2017, 27, 1034-1045.
28. Zang, X.; Thompson, R. H.; AI-Ahmadie, H. A.; Serio, A. M.; Reuter, V. E.; Eastham, J. A.; Scardino, P. T.; Sharma, P.; Allison, J. P. B7-H3 and B7× are highly expressed in human prostate cancer and associated with disease spread and poor outcome. Proc. Natl. Acad. Sci. USA 2007, 104, 19458-19463.
29. Roth, T. J.; Sheinin, Y.; Lohse, C. M.; Kuntz, S. M.; Frigola, X.; Inman, B. A.; Krambeck, A. E.; McKenney, M. E.; Karnes, R. J.; Blute, M. L.; et al. B7-H3 ligand expression by prostate cancer: A novel marker of prognosis and potential target for therapy. Cancer Res. 2007, 67, 7893-7900.
30. Crispen, P. L.; Sheinin, Y.; Roth, T. J.; Lohse, C. M.; Kuntz, S. M.; Frigola, X.; Thompson, R. H.; Boorjian, S. A.; Dong, H.; Leibovich, B. C.; et al. Tumor cell and tumor vasculature expression of B7-H3 predict survival in clear cell renal cell carcinoma. Clin. Cancer Res. 2008, 14, 5150-5157.
31. Zang, X.; Sullivan, P. S.; Soslow, R. A.; Waitz, R.; Reuter, V. E.; Wilton, A.; Thaler, H. T.; Arul, M.; Slovin, S. F.; Wei, J.; et al. Tumor associated endothelial expression of B7-H3 predicts survival in ovarian carcinomas. Mod. Pathol. 2010, 23, 1104-1112.
32. Lemke, D.; Pfenning, P. N.; Sahm, F.; Klein, A. C.; Kempf, T.; Warnken, U.; Schnolzer, M.; Tudoran, R.; Weller, M.; Platten, M.; et al. Costimulatory protein 41gB7H3 drives the malignant phenotype of glioblastoma by mediating immune escape and invasiveness. Clin. Cancer Res. 2012, 18, 105-117.
33. Wang, L.; Zhang, Q.; Chen, W.; Shan, B.; Ding, Y.; Zhang, G.; Cao, N.; Liu, L.; Zhang, Y. B7-H3 is overexpressed in patients suffering osteosarcoma and associated with tumor aggressiveness and metastasis. PLoS ONE 2013, 8, e70689.
34. Yamato, I.; Sho, M.; Nomi, T.; Akahori, T.; Shimada, K.; Hotta, K.; Kanehiro, H.; Konishi, N.; Yagita, H.; Nakajima, Y. Clinical importance of B7-H3 expression in human pancreatic cancer. Br. J. Cancer 2009, 101, 1709-1716.
35. Gregorio, A.; Corrias, M. V.; Castriconi, R.; Dondero, A.; Mosconi, M.; Gambini, C.; Moretta, A.; Moretta, L.; Bottino, C. Small round blue cell tumours: Diagnostic and prognostic usefulness of the expression of B7-H3 surface molecule. Histopathology2008, 53, 73-80.
36. Zhou, Z.; Luther, N.; Ibrahim, G. M.; Hawkins, C.; Vibhakar, R.; Handler, M. H.; Souweidane, M. M. B7-H3, a potential therapeutic target, is expressed in diffuse intrinsic pontine glioma. J. Neuro-Oncol. 2013, 111, 257-264.
37. Calabro, L.; Sigalotti, L.; Fonsatti, E.; Bertocci, E.; Di Giacomo, A. M.; Danielli, R.; Cutaia, O.; Colizzi, F.; Covre, A.; Mutti, L.; et al. Expression and regulation of B7-H3 immunoregulatory receptor, in human mesothelial and mesothelioma cells: Immunotherapeutic implications. J. Cell. Physiol. 2011, 226, 2595-2600.
38. Stern, L. A.; Lown, P. S.; Kobe, A. C.; Abou-Elkacem, L.; Willmann, J. K.; Hackel, B. J. Cellular-Based Selections Aid Yeast-Display Discovery of Genuine Cell-Binding Ligands: Targeting Oncology Vascular Biomarker CD276. ACS Comb. Sci. 2019, 21, 207-222.
39. Shen, B. Q.; Xu, K.; Liu, L.; Raab, H.; Bhakta, S.; Kenrick, M.; Parsons-Reponte, K. L.; Tien, J.; Yu, S. F.; Mai, E.; et al. Conjugation site modulates the in vivo stability and therapeutic activity of antibody-drug conjugates. Nat. Biotechnol. 2012, 30, 184-189.
40. Wall, A.; Wills, A. G.; Forte, N.; Bahou, C.; Bonin, L.; Nicholls, K.; Ma, M. T.; Chudasama, V.; Baker, J. R. One-pot thiol-amine bioconjugation to maleimides: Simultaneous stabilisation and dual functionalisation. Chem. Sci. 2020, 11, 11455-11460.
41. Lim, S. B.; Rubinstein, I.; Onyuksel, H. Freeze drying of peptide drugs self-associated with long-circulating, biocompatible and biodegradable sterically stabilized phospholipid nanomicelles. Int. J. Pharm. 2008, 356, 345-350.
42. Ravasco, J. M. J. M.; Faustino, H.; Trindade, A.; Gois, P. M. P. Bioconjugation with Maleimides: A Useful Tool for Chemical Biology. Chem. A Eur. J. 2019, 25, 43-59.
43. Fontaine, S. D.; Reid, R.; Robinson, L.; Ashley, G. W.; Santi, D. V. Long-term stabilization of maleimide-thiol conjugates. Bioconjugate Chem. 2015, 26, 145-152.
44. Abou-Saleh, R. H.; Peyman, S. A.; Johnson, B. R.; Marston, G.; Ingram, N.; Bushby, R.; Coletta, P. L.; Markham, A. F.; Evans, S. D. The influence of intercalating perfluorohexane into lipid shells on nano and microbubble stability. Soft Matter 2016, 12, 7223-7230.
45. Abou-Saleh, R. H.; Swain, M.; Evans, S. D.; Thomson, N. H. Poly(ethylene glycol) lipid-shelled microbubbles: Abundance, stability, and mechanical properties. Langmuir 2014, 30, 5557-5563.
46. Ferrara, K. W.; Borden, M. A.; Zhang, H. Lipid-shelled vehicles: Engineering for ultrasound molecular imaging and drug delivery. Acc. Chem. Res. 2009, 42, 881-892.
47. Borden, M. A.; Martinez, G. V.; Ricker, J.; Tsvetkova, N.; Longo, M.; Gillies, R. J.; Dayton, P. A.; Ferrara, K. W. Lateral phase separation in lipid-coated microbubbles. Langmuir 2006, 22, 4291-4297.
48. Winther, J. R.; Thorpe, C. Quantification of thiols and disulfides. Biochim. Biophys. Acta (BBA)-Gen. Subj. 2014, 1840, 838-846.
49. Kalia, J.; Raines, R. T. Catalysis of imido group hydrolysis in a maleimide conjugate. Bioorganic Med. Chem. Lett. 2007, 17, 6286-6289.
50. Takalkar, A. M.; Klibanov, A. L.; Rychak, J. J.; Lindner, J. R.; Ley, K. Binding and detachment dynamics of microbubbles targeted to P-selectin under controlled shear flow. J. Control. Release 2004, 96, 473-482.
51. Ahmed, M.; Gustafsson, B.; Aldi, S.; Dusart, P.; Egri, G.; Butler, L. M.; Bone, D.; Dahne, L.; Hedin, U.; Caidahl, K. Molecular Imaging of a New Multimodal Microbubble for Adhesion Molecule Targeting. Cell. Mol. Bioeng. 2019, 12, 15-32.
52. Wang, S.; Hossack, J. A.; Klibanov, A. L. Targeting of microbubbles: Contrast agents for ultrasound molecular imaging. J. Drug Target. 2018, 26, 420-434.
53. Bam, R.; Laffey, M.; Nottberg, K.; Lown, P. S.; Hackel, B. J.; Wilson, K. E. Affibody-Indocyanine Green Based Contrast Agent for Photoacoustic and Fluorescence Molecular Imaging of B7-H3 Expression in Breast Cancer. Bioconjugate Chem. 2019, 30, 1677-1689.
54. Vraka, I.; Panourgias, E.; Sifakis, E.; Koureas, A.; Galanis, P.; Dellaportas, D.; Gouliamos, A.; Antoniou, A. Correlation Between Contrast-enhanced Ultrasound Characteristics (Qualitative and Quantitative) and Pathological Prognostic Factors in Breast Cancer. Vivo 2018, 32, 945-954.
55. Navarro-Becerra, J. A.; Song, K. H.; Martinez, P.; Borden, M. A. Microbubble Size and Dose Effects on Pharmacokinetics. ACS Biomater. Sci. Eng. 2022, 8, 1686-1695.
56. Ergen, C.; Heymann, F.; Al Rawashdeh, W. E.; Gremse, F.; Bartneck, M.; Panzer, U.; Pola, R.; Pechar, M.; Storm, G.; Mohr, N.; et al. Targeting distinct myeloid cell populations in vivo using polymers, liposomes and microbubbles. Biomaterials 2017, 114, 106-120.
57. van Hoeve, W.; de Vargas Serrano, M.; Te Winkel, L.; Forsberg, F.; Dave, J. K.; Sarkar, K.; Wessner, C. E.; Eisenbrey, J. R. Improved Sensitivity of Ultrasound-Based Subharmonic Aided Pressure Estimation Using Monodisperse Microbubbles. J. Ultrasound Med. 2021, 41, 1781-1789.
58. Garg, S.; Thomas, A. A.; Borden, M. A. The effect of lipid monolayer in-plane rigidity on in vivo microbubble circulation persistence. Biomaterials 2013, 34, 6862-6870.
59. Wright, T. A.; Rahman, M. S.; Bennett, C.; Johnson, M. R.; Fischesser, H.; Ram, N.; Tyler, A.; Page, R. C.; Konkolewicz, D. Hydrolytically Stable Maleimide-End-Functionalized Polymers for Site-Specific Protein Conjugation. Bioconjugate Chem. 2021, 32, 2447-2456.
60. Badescu, G.; Bryant, P.; Swierkosz, J.; Khayrzad, F.; Pawlisz, E.; Farys, M.; Cong, Y.; Muroni, M.; Rumpf, N.; Brocchini, S.; et al. A new reagent for stable thiol-specific conjugation. Bioconjugate Chem. 2014, 25, 460-469.
61. Bam, R.; Lown, P. S.; Stern, L. A.; Sharma, K.; Wilson, K. E.; Bean, G. R.; Lutz, A. M.; Paulmurugan, R.; Hackel, B. J.; Dahl, J.; et al. Efficacy of Affibody-Based Ultrasound Molecular Imaging of Vascular B7-H3 for Breast Cancer Detection. Clin. Cancer Res. 2020, 26, 2140-2150.
62. Jugniot, N.; Bam, R.; Paulmurugan, R. Expression and purification of a native Thy1-single-chain variable fragment for use in molecular imaging. Sci. Rep. 2021, 11, 23026.
63. Vuković, L.; Khatib, F. A.; Drake, S. P.; Madriaga, A.; Brandenburg, K. S.; Král, P.; Onyuksel, H. Structure and dynamics of highly PEG-ylated sterically stabilized micelles in aqueous media. J. Am. Chem. Soc. 2011, 133, 13481-13488.
64. Püspöki, Z.; Sage, D.; Ward, J. P.; Unser, M. SpotCaliper: Fast wavelet-based spot detection with accurate size estimation. Bioinformatics 2016, 32, 1278-1280.
65. Gage, G. J.; Kipke, D. R.; Shain, W. Whole animal perfusion fixation for rodents. J. Vis. Exp. 2012, e3564.
66. Abou-Elkacem, L.; Wang, H.; Chowdhury, S. M.; Kimura, R. H.; Bachawal, S. V.; Gambhir, S. S.; Tian, L.; Willmann, J. K. Thy1-Targeted Microbubbles for Ultrasound Molecular Imaging of Pancreatic Ductal Adenocarcinoma. Clin. Cancer Res. 2018, 24, 1574-1585.

What is claimed is:

1. A method of producing a phospholipid-ligand bioconjugate, the method comprising:
contacting:
a) a phospholipid polymer comprising a maleimide-containing functional group, with
b) a ligand comprising a C terminal cysteine residue.

2. The method of claim 1, wherein the phospholipid is 2-distearoyl-sn-glycero-3-phosphoethanolamine-N-[maleimide(polyethylene glycol)-2000] (ammonium salt) (DSPE-PEG[2000]-Mal).

3. The method of claim 1, wherein the ligand is selected from an affibody, an antibody, a VHH antibody, a single chain variable fragment, and a diabody.

4. The method of claim 3, wherein the ligand binds to a surface protein expressed on an endothelial or a cancer cell.

5. The method of claim 4, wherein the surface protein is B7-H3.

6. The method of claim 1, wherein the ligand is AC12 according to SEQ ID NO: 5.

7. The method of claim 1, wherein the C terminal cysteine residue is preceded by a pentaglycine bridge.

8. The method of claim 1, wherein the phospholipid is contacted with the ligand in a 20 to 1 ratio (20 phospholipid to 1 ligand).

9. The method of claim 1, further comprising heating the phospholipid to greater than 50° C. for at least 3 hours and reducing the temperature of the phospholipid to room temperature for at least 1 hour prior to the contacting step.

10. The method of claim 1, wherein the contacting is performed under neutral pH conditions.

11. A method of producing a target microbubble, the method comprising contacting a micelle comprising the phospholipid-ligand conjugate of claim 1 with:
a) a phospholipid liposome; and
b) an inert gas.

12. The method of claim 11, wherein the phospholipid is 1,2-dipalmitoyl-sn-glycero-3-phosphocholine (DPPC).

13. The method of claim 11, wherein the inert gas is perfluorobutane gas.

14. The method of claim 11, wherein the targeted microbubble comprises 1-10 mole percent of the phospholipid-ligand conjugate.

15. The method of claim 11, wherein the targeted microbubble comprises 90-99 mole percent of the phospholipid.

16. The method of claim 11, wherein the phospholipid liposome is 100 nm in diameter.

17. The method of claim 11, wherein the targeted microbubble has diameter of 0.5 μm to 5 μm.

18. The method of claim 11, wherein the contacting is performed in a microfluidic device.

19. The method of claim 11, wherein the contacting is performed in a mechanical agitation device.

20. The method of claim 11, further comprising contacting the micelle comprising the phospholipid-ligand bioconjugate with a second micelle comprising a phospholipid-therapeutic agent bioconjugate, wherein the therapeutic agent is selected from the group consisting of a chemotherapeutic agent, a toxin, a radioactive isotope, a kinase inhibitor, an immunomodulator, and a hormone blocker.
